SEQUENCE LISTING (1 sequence)

SEQ ID NO: 1 - protein, 406 aa, Homo sapiens [sequence omitted]

(19) United States
(12) Patent Application Publication
(10) Pub. No.: US 2009/0191179 A1
(43) Pub. Date: Jul. 30, 2009

(54) Use of factor VIIa analogues with increased activity

(75) Inventors: Dorthe Viuff, Frederikssund (DK); Mirella Ezban, Copenhagen O (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk HealthCare A/G, Zurich (CH)

(21) Appl. No.: 12/321,192

(22) Filed: Jan. 16, 2009

Related U.S. Application Data
(63) Continuation-in-part of application No. PCT/EP2007/057244, filed on Jan. 13, 2007.
(60) Provisional application No. 60/835,361, filed on Aug. 3, 2006.
(60) Provisional application No. 60/835,356, filed on Aug. 3, 2006.

(30) Foreign Application Priority Data
Jul. 17, 2006 (EP) 06117283.9
Jul. 17, 2006 (EP) 06117284.7
Jul. 6, 2007 (EP) 07111940.8

(51) Int. Cl. A61K 38/48; A61P 7/00

(52) U.S. Cl. 424/94.64

(57) ABSTRACT

The invention relates to methods for the treatment of a severely bleeding subject.

### CROSS-REFERENCE TO RELATED APPLICATIONS

This application is a Continuation-in-Part under 35 U.S.C. 111(a) (i.e. claims priority under 35 U.S.C. 120) of International Patent Application PCT/EP2007/057244 (published as WO 2008/009634), filed Jul. 13, 2007, and claims priority under 35 U.S.C. § 119 of European Patent Applications 06117284.7, filed Jul. 17, 2006, and 06117283.9, filed Jul. 17, 2006 and 07111940.8, filed Jul. 6, 2007; this application further claims priority of U.S. Provisional Applications 60/835,361 and 60/835,356, filed Aug. 3, 2006.

### FIELD OF THE INVENTION

The present invention relates to methods for treatment of severely bleeding episodes in a subject, including the prevention of, or minimizing severity of, late complications in bleeding episodes in such subjects with severely bleedings.

### BACKGROUND OF THE INVENTION

Haemostasis is a complex physiological process which ultimately results in the arrest of bleeding. This is dependent on the proper function of three main components: blood vessels (especially the endothelial lining), coagulation factors, and platelets. Once a haemostatic plug is formed, the timely activation of the fibrinolytic system is equally important to prevent further unnecessary haemostatic activation. Any malfunction of this system (due to a reduced number, or molecular dysfunction, of the haemostatic components or increased activation of the fibrinolytic components) may lead to clinical bleeding such as, e.g., haemorrhagic diathesis of varying severity.

In most physiological situations, haemostasis is triggered by the interaction of circulating activated coagulation factor VII (FVIIa) with tissue factor (TF) subsequent to exposure of TF at the site of an injury. Endogenous FVIIa becomes proteolytically active only after forming a complex with TF. Normally, TF is expressed in the deep layers of the vessel wall and is exposed following injury. This ensures a highly localized activation of coagulation and prevents disseminated coagulation. TF also seems to exist in a non-active form, so-called encrypted TF. The regulation of encrypted versus active TF is still unknown.

Activated recombinant wild type human factor VII (rFVIIa) is indicated for the treatment of bleeding episodes in haemophilia A or B patients with inhibitors to Factor VIII or Factor IX. When given in high (pharmacological) doses, rFVIIa can bind independently of TF to activated platelets and initiate local thrombin generation which is important for the formation of the initial haemostatic plug.

Uncontrolled bleeding is a major cause of death in severe bleeding conditions, such as trauma and there is a need in the art for improved methods and compositions for treatment of these severe bleedings, as well as for prevention and attenuation of late complications that result from severe bleedings or from blood transfusion.

### SUMMARY OF THE INVENTION

The invention provides the use of a Factor VII polypeptide having increased activity compared to wild-type Factor VIIa for the manufacture of a medicament for treatment of bleeding episodes in a subject with severe bleedings.

In one aspect the present invention relates to a method for the treatment of bleeding episodes in a subject with severe bleedings, the method comprising administering to a subject in need of said treatment an effective amount for said treatment of a Factor VII polypeptide having increased activity compared to wild-type Factor VIIa.

The invention also provides methods for preventing or attenuating the symptoms of bleeding episodes in a subject with severe bleedings, which are carried out by administering to a subject an effective amount for said preventing or attenuating of a Factor VII polypeptide having increased activity compared to wild-type Factor VIIa.

In some embodiments, the effective amount comprises at least about 1 μg/kg of a Factor VII polypeptide, such as at least about 10 μg/kg, such as at least about 20 μg/kg, such as at least about 40 μg/kg, such as at least about 80 μg/kg of a Factor VII polypeptide having increased activity compared to wild-type Factor VIIa. In some embodiments, the effective amount comprises at least about 100 μg/kg of a Factor VII polypeptide having increased activity compared to wild-type Factor VIIa. In some embodiments, a first amount of at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa is administered at the start of treatment, and a second amount of at least about 1 μg/kg of a Factor VII polypeptide, such as at least about 10 μg/kg, such as at least about 20 μg/kg, such as at least about 40 μg/kg, such as at least about 80 μg/kg of Factor VII polypeptide having increased activity compared to wild-type Factor VIIa is administered to the subject one or more hours after the start of treatment. In some embodiments, a third amount of at least about 1 μg/kg of a Factor VII polypeptide, such as at least about 10 μg/kg, such as at least about 20 μg/kg, such as at least about 40 μg/kg, such as at least about 80 μg/kg of Factor VII polypeptide having increased activity compared to wild-type Factor VIIa is administered at a later time, such as, e.g. least about one hour after the start of the second treatment. It is to be understood that the exact amount of FVII polypeptide administered will vary depending on the specific increase in activity of the Factor VII polypeptide having increased activity compared to wild-type Factor VIIa and also depending on specific severe bleeding indication being treated.

In some embodiments, the method further comprises administering to the subject a second coagulation agent in an amount that augments the treatment by said Factor VII polypeptide having increased activity compared to wild-type Factor VIIa. Preferably, the second coagulation agent is a coagulation factor (including, without limitation, Factor V, Factor VIII, Factor IX, Factor X, Factor XI, Factor XIII, Fibrinogen, thrombin, TAFI; an anti-fibrinolytics such as, e.g., PAI-1, aprotinin, epsilon-aminocaproic acid or tranexamic acid, various antithrombotic treatments, as well as transfusions with platelet, RBC, FFP, oxygen carriers, the various bypassing agents and fluid therapies (colloids/crystalloids), or any combination thereof.

The present invention also provides a kit of parts for treatment of bleeding episodes in subjects with severe bleedings, comprising
(i) A medicament comprising a Factor VII polypeptide having increased activity compared to wild-type Factor VIIa; and
(ii) Instructions for use describing that:
a. A first dose containing at least about 1 μg/kg of a Factor VII polypeptide, such as at least about 10 μg/kg, such as at least about 20 μg/kg, such as at least about 40 μg/kg, such as at least about 80, such as at least about 100 μg/kg Factor VII polypeptide having increased activity compared to wild-type Factor VIIa, should be administered at the start of treatment;
b. Optionally, a second dose containing at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa should be administered one to 24 hours after the start of treatment.

The present invention also provides method for treating bleeding episodes in a subject with severe bleedings in a majority of subjects with severe bleedings, said method comprising (i) administering to a group of subjects with severe bleedings an effective amount for said treatment of Factor VII polypeptide having increased activity compared to wild-type Factor VIIa; and (ii) observing an improvement in one or more clinical parameters of said severe bleeding episode among said group of subjects relative to the level of said clinical parameters that would have been expected in the same group of subjects who had not received said Factor VII polypeptide having increased activity compared to wild-type Factor VIIa.

### DETAILED DESCRIPTION OF THE INVENTION

Massive haemorrhage after traumatic injury, or complicated surgical procedures, is frequently a combination of surgical and coagulopathic bleeding. Surgical bleeds originate from a recognizable source at the site of surgery, or trauma, whereas coagulopathic bleeding results from impaired thrombin generation. Coagulopathy develops early after injury and correlates to the severity of the injury. The mechanisms behind coagulopathy in severe bleedings such as trauma are complex and multifactorial including dilution coagulopathy, hypothermia, acidosis, hyperfibrinolysis, and consumption coagulopathy.

One of the patent examples of the present invention demonstrates the impact of low pH (in vitro model of acidosis) and temperature (in vitro model of hypothermia) as well as crystalloid and colloid hemodilution on the effect of wild type recombinant FVIIa (rwtFVIIa) and a Factor VII polypeptide having increased activity compared to wild-type Factor VIIa, such as V158D/E296V/M298Q-FVIIa.

In preclinical and in vitro studies, haemodilution is a model which mimics the clinical situation of bleeding and resuscitation induced dilution of red blood cells and coagulation factors. The degree of dilution is reflected in the haemoglobin level and in the amount of fluids administered.

The reference interval for haemoglobin varies a bit from laboratory to laboratory, but is generally in the range of 12-16 g/dl for women and 14-18 g/dl for men. Severe bleeding patients such as trauma patients are a very heterogenous population and their haemoglobin levels vary greatly, depending on the degree of bleeding and the fluid and blood product replacement therapy.

Anticoagulants have shown to be effective in prevention and treatment of thromboembolic events. Major bleeding complications are rare and may often be managed by temporary discontinuation of the drug, however, in some cases of acute serious bleeding, an effective and instant haemostatic intervention may be needed. Thus, in one aspect the present invention relates to the use of a Factor VII polypeptide having increased activity compared to wild-type Factor VIIa, such as V158D/E296V/M298Q-FVIIa for the manufacture of a medicament for the treatment of bleeding episodes in a subject pre-treated with an anti-coagulant. In a further aspect the present invention relates to the use of a Factor VII polypeptide having increased activity compared to wild-type Factor VIIa, such as V158D/E296V/M298Q-FVIIa for the for the treatment of bleeding episodes in a subject pretreated with an anticoagulant. In one embodiments the anticoagulant is selected form the list consisting of Vitamin-K antagonists, such as Warfarin, phenprocoumon and acenocoumarol; heparin, low-molecular-weight heparins (LMWHs), danaparoid, fondaparinux, lepirudin, bivalirudin, argatroban, megalatran, ximelagatran, aspirin, clopidogrel, abciximab, tirofiban, and eptifibatide. Recombinant wild type FVIIa has earlier shown to have no effect as an antidote to LMW Heparin treatment in a rabbit ear bleeding model (Chan S et al., Thromb Haem 2003; 1:760-765). However, the inventors of the present invention have investigated the effect of recombinant wtFVIIa and Factor VII polypeptide having increased activity compared to wild-type Factor, such as V158D/E296V/M298Q-FVIIa in blood mimicking anti-coagulant conditions and have found that Factor VII polypeptides having increased activity compared to wild-type Factor, such as V158D/E296V/M298Q-FVIIa ex vivo is significantly superior to equimolar concentration of recombinant wtFVIIa.

The term “acidosis” as used herein means an increased acidity (i.e. hydrogen ion concentration) of blood plasma in a subject as compared to the normal acidity. Generally acidosis is said to occur when arterial pH falls below 7.35. Thus, in one embodiment, pH in the subject having a severe bleeding is below 7.35.

The term “hypothermia” as used herein means a physical condition in a subject in which the body's core temperature drops below normal temperature in this subject. Generally hypothermia is said to occur when the body's core temperature drops below 35 degree Celsius.

The term “severely bleeding” refers to a condition in a subject in which this subject has a bleeding which requires:
1) ongoing fluid replacement therapy at a rate of at least 1 L per hour; and/or
2) requirement for Red Blood Cell replacement therapy; and/or
3) which results in one or more of the physical conditions in said subject selected from the list consisting of:
   a) measurable acidosis;
   b) hypotension with a systolic blood pressure lower than 100 mmHg;
   c) haemoglobin level 4 g/dl lower than the normal level for said subject.

In one embodiment, the subject with severe bleeding experience acidosis with a pH lower than 7.40, such as lower than 7.35, such as lower than 7.25, such as lower than 7.15, such as lower than 7.05, such as lower than 6.95, such as lower than 6.85.

In one embodiment, the subject has a severe bleeding due to trauma.

In one embodiment, the subject has a severe bleeding due to a blunt trauma.

The term “blunt trauma” as used herein, refers to injuries caused by non-penetrating forces such as motor vehicle crashes, falls, or assaults with a blunt object.

In one embodiment, the subject has a severe bleeding due to a penetrating trauma.

The term “Penetrating trauma” as used herein, refers to injuries that disrupts the body surface and extends into underlying tissue or body cavity, such as stab or gunshot wounds.

In one embodiment, the subject has a severe bleeding due to a burn trauma.

Typical patients having experienced burn trauma need an excision of tissue, such as an excision of tissue of 5% or more of total body surface area (TBSA), such as 10 or more of TBSA. Typical patients also experience microvascular bleedings.

Excision of burn wounds is frequently associated with a large volume of blood loss requiring allogeneic blood transfusion. Thus in some embodiments the administration of the Factor VII polypeptide having increased activity compared to wild-type Factor VIIa reduces blood transfusion requirement in burn patients undergoing excision and skin grafting.

In some embodiments, the initial administering step is carried out within 5 hours of the occurrence of the traumatic burn. In some embodiments, the initial administering step is carried out immediately before start of excision surgery. In some embodiments, an administering step is carried out after excision surgery, such as repeated 60 minutes after.

In one embodiment, the subject with severe bleeding has a requirement for red blood cell replacement therapy.

In one embodiment, the subject with severe bleeding experience hypotension with a systolic blood pressure lower than 100 mmHg, such as lower than 95 mmHg, such as lower than 90 mmHg.

In one embodiment, the subject with severe bleeding experience hypothermia with a temperature below 37 degree Celsius, such as below 36 degree celsius, such as below 35 degree celsius, such as below 34 degree celsius, such as below 33 degree celsius, such as below 32 degree celsius, such as below 31 degree celsius, such as below 30 degree celsius.

In one embodiment, the subject with severe bleeding experience pre-treatment haemoglobin level 4 g/dl lower than the normal level for said subject, such as 5 g/dl lower than the normal level for said subject, such as 6 g/dl lower than the normal level for said subject, such as 7 g/dl lower than the normal level for said subject, such as 8 g/dl lower than the normal level for said subject, such as 9 g/dl lower than the normal level for said subject, such as 10 g/dl lower than the normal level for said subject, such as 11 g/dl lower than the normal level for said subject, such as 12 g/dl lower than the normal level for said subject, such as 13 g/dl lower than the normal level for said subject.

In one embodiment, the subject with severe bleeding experience pre-treatment haemoglobin level lower than 14 g/dl, such as a pre-treatment haemoglobin level lower than 12 g/dl, such as a pre-treatment haemoglobin level lower than 10 g/dl, such as a pre-treatment haemoglobin level lower than 8 g/dl, such as a pre-treatment haemoglobin level lower than 6 g/dl, such as a pre-treatment haemoglobin level lower than 4 g/dl, such as a pre-treatment haemoglobin level lower than 3 g/dl, such as a pre-treatment haemoglobin level lower than 2 g/dl.

In one embodiment, the subject with severe bleeding requires fluid replacement therapy of 1000 ml crystalloids and/or colloids, such as a fluid replacement therapy of 1500 ml crystalloids or colloids, such as a fluid replacement therapy of 2000 ml crystalloids or colloids, such as a fluid replacement therapy of 2500 ml crystalloids or colloids, such as a fluid replacement therapy of 3000 ml crystalloids or colloids, such as a fluid replacement therapy of 3500 ml crystalloids or colloids, such as a fluid replacement therapy of 4000 ml crystalloids or colloids, such as a fluid replacement therapy of 4500 ml crystalloids or colloids, such as a fluid replacement therapy of 5000 ml crystalloids or colloids, such as a fluid replacement therapy of 6000 ml crystalloids or colloids.

Colloid and Crystalloid solutions are widely used in fluid replacement therapy of critically bleeding patients. There are several choices of colloid and crystalloid, which are well known by the persons skilled in the art. Examples includes, concentrated albumin, plasma products, dextrans, hetastarch, synthetically derived colloids.

Examples of isotonic crystalloids includes lactated Ringer's(see below), Plasmalyte-ATM, and Normosol-RTM. Examples of hypertonic crystalloids includes 3.0%, 7.0% and 7.5% saline and 5% glucose added to balanced electrolyte solutions or maintenance solutions.

In another embodiment the subject with severe bleeding requires blood product therapy. Blood products include whole blood, plasma, platelet concentrates and cryoprecipitatee.

In a first aspect the present invention relates to the use of a Factor VII polypeptide having increased activity compared to wild-type Factor VIIa for the manufacture of a medicament for the treatment of bleeding episodes in a subject with severe bleedings.

In a second aspect the present invention relates to a kit of parts for the treatment of bleeding episodes in a subject with severe bleedings, comprising
(i) A medicament comprising a Factor VII polypeptide having increased activity compared to wild-type Factor VIIa; and
(ii) Instructions for Use describing that:
a. A first dose containing at least about 1 μg/kg of a Factor VII polypeptide, such as at least about 10 μg/kg, such as at least about 20 μg/kg, such as at least about 40 μg/kg, such as at least about 80, such as at least about 100 μg/kg Factor VII polypeptide having increased activity compared to wild-type Factor VIIa, should be administered at the start of treatment;
b. Optionally, a second dose containing at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa should be administered one to 24 hours after the start of treatment.

In a third aspect the present invention relates to a method for treating bleeding episodes in a subject with thrombocytopenia, the method comprising administering to a subject in need of said treatment an effective amount for said treatment of a Factor VII polypeptide having increased activity compared to wild-type Factor VIIa.

In a further aspect the present invention relates to a method for preventing treating bleeding episodes in a subject with thrombocytopenia, the method comprising intentionally administering to a subject in need of said treatment an effective amount for said treatment of Factor VII polypeptide having increased activity compared to wild-type Factor VIIa, such as V158D/E296V/M298Q-FVIIa for the treatment of bleeding episodes in a subject with severe bleedings.

In a further aspect the present invention relates to a method for the treatment of bleeding episodes in a subject with severe bleedings in a majority of subjects with severe bleedings, said method comprising (i) administering to a group of subjects with severe bleedings an effective amount for said treatment of Factor VII polypeptide having increased activity compared to wild-type Factor VIIa; and (ii) observing an improvement in one or more clinical parameters of said bleeding episode among said group of subjects relative to the level of said clinical parameters that would have been expected in the same group of subjects who had not received said Factor VII polypeptide having increased activity compared to wild-type Factor VIIa.

One aspect of the present invention relates to the use of Factor VII polypeptide having increased activity compared to wild-type Factor VIIa in reducing or avoiding perioperative blood transfusion requirement in subjects with severe bleedings undergoing surgery. In one series of embodiments, treatment of subjects according to the invention results in reduction in a perioperative blood transfusion requirement by 10%, such as 20%, such as 40%, such as 60%, such as 80%, such as 100%.

The term “bleeding episodes” is meant to include uncontrolled and excessive bleeding. Bleeding episodes may be a major problem both in connection with surgery, such as but not limited to cardiac, liver, orthopaedic, or prostate surgery and other forms of tissue damage or it may be spontaneous. Uncontrolled and excessive bleeding may occur in subjects having a coagulation or bleeding disorder. As used herein the term “bleeding disorder” reflects any defect, congenital, acquired or induced, of cellular or molecular origin that is manifested in bleedings. Examples are clotting factor deficiencies (e.g. haemophilia A and B or deficiency of coagulation Factors XI or VII), clotting factor inhibitors, defective platelet function, or von Willebrand's disease.

Excessive bleedings also occur in subjects with a normally functioning blood clotting cascade (no clotting factor deficiencies or -inhibitors against any of the coagulation factors) and may be caused by a defective platelet function, thrombocytopenia or von Willebrand's disease. In such cases, the bleedings may be likened to those bleedings caused by haemophilia because the haemostatic system, as in haemophilia, lacks or has abnormal essential clotting “compounds” (such as von Willebrand factor protein) that causes major bleedings.

In subjects who experience extensive tissue damage in association with surgery or vast trauma, the normal haemostatic mechanism may be overwhelmed by the demand of immediate haemostasis and they may develop bleeding in spite of a normal haemostatic mechanism. Achieving satisfactory haemostasis also is a problem when bleedings occur in organs such as the brain, inner ear region and eyes with limited possibility for surgical haemostasis. The same problem may arise in the process of taking biopsies from various organs (liver, lung, tumour tissue, gastrointestinal tract) as well as in laparoscopic surgery. Common for all these situations is the difficulty to provide haemostasis by surgical techniques (sutures, clips, etc.) which also is the case when bleeding is diffuse (haemorrhagic gastritis and profuse uterine bleeding). Acute and profuse bleedings may also occur in subjects on anticoagulant therapy in whom a defective haemostasis has been induced by the therapy given. Such subjects may need surgical interventions in case the anticoagulant effect has to be counteracted rapidly. Radical retropubic prostatectomy is a commonly performed procedure for subjects with localized prostate cancer. The operation is frequently complicated by significant and sometimes massive blood loss. The considerable blood loss during prostatectomy is mainly related to the complicated anatomical situation, with various densely vascularized sites that are not easily accessible for surgical haemostasis, and which may result in diffuse bleeding from a large area. Another situation that may cause problems in the case of unsatisfactory haemostasis is when subjects with a normal haemostatic mechanism are given anticoagulant therapy to prevent thromboembolic disease. Such therapy may include heparin, other forms of proteoglycans, warfarin or other forms of vitamin K-antagonists as well as aspirin and other platelet aggregation inhibitors.

There is a particularly high risk of spontaneous bleeding once the platelet count drops below 10 million per ml. Thus, in one embodiment of the invention, the subject having a severe bleeding also has thrombocytopenia.

In one embodiment, the bleeding is intracranial. In one embodiment, the bleeding is intracerebral haemorrhage (ICH).

Intracerebral haemorrhage (ICH) is a neurologic condition that occurs spontane-ous and results in blood collecting in the intraparenchymal brain tissue. The results of an ICH have been demonstrated to result in significant morbidity and mortality. In recent years ICH has been shown to increase in volume in the hours following the initial insult. The reason for the increase is unclear, but it is thought to be either through a continuous oozing of the original haematoma or through a complex process of rebleeds.

Days after the initial insult a zone of oedema can be identified on CT scans—surrounding the blood in the haematoma. The mechanism for oedema generation is also poorly understood but may be due to a combination of an inflammatory reaction in the tissue surrounding the clot as well as a direct mass effect of the clot exerting pressure on surrounding brain tissue. The impact of the isolated oedema can be significant but not evaluated in the context of significant haemorrhage but can have effects on the volume of compromised brain tissue after an ICH which has been estimated to be up to 3 times the actual volume of the haematoma. The importance of overall effected tissue volume would appear to be one of the strongest predictors of outcome after ICH. Thus there is clinical interest in reducing any haemorrhage expansion and in reducing and/or minimiz-ing the total lesion volume (blood and resulting oedema). In some embodiments, the initial administering step is carried out within 4 hours of the occurrence of the ICH. In one series of embodiments, the excessive bleeding is caused by spontaneous ICH; in another it is caused by traumatic ICH.

In one embodiment the treatment with an effective amount of a Factor VII polypeptide having increased activity compared to wild-type Factor VIIa, such as V158D/E296V/M298Q-FVIIa is for preventing or attenuating haemorrhage growth, and/or oedema generation in an ICH patient leading to improved outcome. In one embodiment the treatment with an effective amount of a Factor VII polypeptide having increased activity compared to wild-type Factor VIIa is for preventing or attenuating haemorrhage growth, and/or oedema generation following ICH in patients who have received antiplatelet therapy prior to occurrence of the ICH.

In one embodiment the bleeding is due to antiplatelet therapy, such as therapy that inhibits platelet aggregation.

In one embodiment, the bleeding is gastrointestinal, such as bleeding from the upper gastrointestinal tract due to haemorrhagic gastritis. In one embodiment, the bleeding is from esophageal varices. In one embodiment, the bleeding is from Acute Abdominal Aneurysm (MA).

In one embodiment, the bleeding is urogenital.

In one embodiment, the bleeding is retinal.

In one embodiment of the invention, the subject with severe bleeding has haemophilia. In another embodiment, the subject with severe bleeding has haemophilia with acquired inhibitors. In another embodiment, the subject with severe bleeding has von Willebrand's disease. In another embodiment, the subject with severe bleeding has severe tissue damage. In another embodiment, the subject with severe bleeding has severe trauma. In another embodiment, the severe bleeding is associated with surgery. In another embodiment, the severe bleeding is associated with laparoscopic surgery. In another embodiment, the severe bleeding is associated with haemorrhagic gastritis or bleeding from esophageal varices. In another embodiment, the severe bleeding is associated with profuse uterine bleeding. In another embodiment, the severe bleeding is occurring in organs with a limited possibility for mechanical haemostasis. In another embodiment, the severe bleeding is occurring in the brain, inner ear region or eyes. In another embodiment, the severe bleeding is associated with the process of taking biopsies. In another embodiment, the severe bleeding is associated with anticoagulant therapy.

In one particular set of embodiments the severe bleeding is in a patient with a normal coagulation system, such as a patient who do not suffer from clotting factor deficiencie (e.g. haemophilia A and B or deficiency of coagulation Factors XI or VII), clotting factor inhibitors, defective platelet function, or von Willebrand's disease.

In the present context, the term “treatment” is meant to include both prevention of an expected severe bleeding, such as in surgery, and regulation of an already occurring severe bleeding, such as in trauma, with the purpose of inhibiting or minimising this severe bleeding. Prophylactic administration of the Factor VII polypeptide having increased activity compared to wild-type Factor VIIa is thus included in the term “treatment”.

The term “subject” as used herein is intended to mean any animal, in particular mammals, such as humans, and may, where appropriate, be used interchangeably with the term “patient”.

The present invention provides methods and compositions that can be used advantageously to treat bleeding episodes in a subject with severe bleedings.

The methods are carried out by administering to this subject with severe bleedings Factor VII polypeptide having increased activity compared to wild-type Factor VIIa, such as V158D/E296V/M298Q-FVIIa, in a manner that is effective for treatment. A manner effective for treatment may comprise administering a predetermined amount of Factor VII polypeptide having increased activity compared to wild-type Factor VIIa, and/or utilizing a particular dosage regimen, formulation, mode of administration, combination with other treatments, and the like. The efficacy of the methods of the invention in treating bleeding episodes in a subject with severe bleedings may be assessed using one or more conventionally used parameters of the immediate consequences of injury and/or late complications. Immediate consequences include, e.g., blood loss and symptoms of shock; while late complications, include, without limitation, Pulmonary embolism (PE), Acute Respiratory Distress Syndrome (ARDS), Disseminated Intravascular Coagulation (DIC), Acute Myocardial Infarction (AMI), Cerebral Thrombosis (CT), Systemic Inflammatory Response Syndrome (SIRS), infections, sepsis, Multiple Organ Failure (MOF), and Acute Lung Injury (ALI), including death caused by one or more of these syndromes.

Thus in some aspects the present invention relates to a method of reducing the risk of immediate consequences of injury and/or late complications the method comprising administering to a subject in need of said treatment an effective amount for said treatment of Factor VII polypeptide having increased activity compared to wild-type Factor VIIa, such as V158D/E296V/M298Q-FVIIa. In one embodiment the present invention relates to a method of reducing the risk of immediate consequences of injury and/or late complications selected from the list consisting of Pulmonary embolism (PE), Acute Respiratory Distress Syndrome (ARDS), Disseminated Intravascular Coagulation (DIC), Acute Myocardial Infarction (AMI), Cerebral Thrombosis (CT), Systemic Inflammatory Response Syndrome (SIRS), infections, sepsis, Multiple Organ Failure (MOF), and Acute Lung Injury (ALI), including death caused by one or more of these syndromes, the method comprising administering to a subject in need of said treatment an effective amount for said treatment of Factor VII polypeptide having increased activity compared to wild-type Factor VIIa, such as V158D/E296V/M298Q-FVIIa. In one embodiment the late complication is Multiple Organ Failure (MOF). In one embodiment the late complication is Acute Respiratory Distress Syndrome (ARDS). In one embodiment the late complication is Acute Lung Injury (ALI). In one embodiment the late complication is sepsis.

Coagulopathy in severe bleedings such as trauma is multifactorial, encompassing coagulation abnormalities resembling DIC, caused by systemic activation of coagulation and fibrinolysis; excessive fibrinolysis, which can be evident on the first day in some severe bleedings subjects, such as trauma subjects; and dilutional coagulopathy, which is caused by excessive fluid administration. Some fluids such as hydroxyethyl starch (HES) preparations may directly compromise coagulation. Massive transfusion syndrome results in depletion of coagulation factors and impairment of platelet function. Hypothermia causes a slower enzyme activity of the coagulation cascade and dysfunctional platelets. Metabolic abnormalities, such as acidosis, also compromise coagulation especially when associated with hypothermia.

Non-limiting examples of subjects in need of treatment according to the invention include those who exhibit one or more of the following:

Coagulation abnormalities resembling DIC, caused by systemic activation of coagulation and fibrinolysis

Excessive fibrinolysis

Dilutional coagulopathy caused by excessive fluid treatment, including, without limitation, a limited number of platelets and/or an impaired platelet function compared to the platelet count and platelet activity of normal pooled blood

Receipt of hydroxyethyl starch (HES) preparations

Hypothermia, a including having body temperature below about 37° C., such as, e.g., below about 36° C., below about 35° C., or below about 34° C.

At least one indication of metabolic abnormalities, including, without limitation, acidosis having a blood pH below about 7.4, below about 7.3, below about 7.2, or below about 7.1.

In one series of embodiments, subjects treated according to the invention are those who require transfusion with whole blood (WB), packed red blood cells (pRBC), or fresh frozen plasma (FFP), such as, e.g., more than about 2 units, 5 units, or more than about 8 units, between the time of their bleeding and the time of administration of Factor VII polypeptide having increased activity compared to wild-type Factor VIIa, such as V158D/E296V/M298Q-FVIIa. A unit of WB typically contains about 450 ml blood and 63 ml of conventional anticoagulant/preservative (having a hematocrit of 36-44%). A unit of pRBC typically contains 200-250 ml of red blood cells, plasma, and conventional anticoagulant/preservative (having a hematocrit of 70-80%).
In one embodiment, the bleeding is due to Postpartum Hemorrhage.

Post partum hemorrhage (PPH) is an excessive bleeding due to inability of the uterus to contract (atony), incompletely delivered placenta (placenta praevia or accreta), uterine rupture, birth canal injury or amniotic fluid embolism. Estimated blood loss in excess of 500 ml within 24 hours of delivery but shifting to more clinically applicable definition of 1000 ml. Severe postpartum hemorrhage (SPPH) is usually defined as a blood loss in excess of 1500 ml, with a drop in hemoglobin concentration of ≧4 g/dl and the need of ≧4 units of blood.

Post partum hemorrhage remains a dangerous obstetrical complication and represents one of the most common reasons for maternal morbidity and mortality worldwide. Life-threatening haemorrhage has been estimated to occur in 1 per 1000 deliveries and it is estimated that 125000 women die worldwide from PPH each year.

The most important complications associated with SPPH include hypovolemic shock, disseminated intravascular coagulopathy (DIC) renal failure, hepatic failure and adult respiratory distress syndrome (ARDS). Another significant cause of morbidity is the loss of fertility secondary to hysterectomy. Factors influencing the outcome are: underestimation of blood loss, late diagnosis of uterine rupture, undiagnosed concealed abruption, late diagnosis of retroperitoneal or intraabdominal bleeding, rapid changes of coagulation events, inability to accurately diagnose the severity of DIC and the lack of ideal hospital facilities.

Many women who develop massive, life-threatening PPH have a combination of ‘coagulopathic’ diffuse bleeding and ‘surgical’ bleeding. While bleeding from larger vessels might be controllable by a surgeon, the ability to control of diffuse bleeding is either limited or, in many cases, not feasible.

Mainstays of management of PPH usually involve effective transfusion therapy, uterotonic medications (oxytocin, ergotamine, prostaglandins) and invasive/surgical methods. Conditions with excessive bleeding, such as uterine rupture, placenta accreta, abruption or uterine atony, may require intensive resuscitation with blood components and coagulation factors. Blood transfusion in such circumstances may be life saving but involves exposing the patient to additional risks. Long established surgical measures include iliac artery ligation and subtotal or total hysterectomy, and newer, less radical measures that may preserve reproductive function, such as angiographic embolization, uterine compression sutures and methods involving uterine tamponade. However, only few centers have access to the equipment or resources necessary to conduct the angiographic embolisation procedure and this intervention may not be feasible in an emergency.

The overall effectiveness of these procedures in stopping haemorrhage and preventing the need for emergency hysterectomy as a final measure to control the bleeding is estimated to be 50%. In cases of intractable bleeding unresponsive to conventional medical therapies and surgical procedures, hysterectomy is usually required as a final measure to control the bleeding. However, peripartum hysterectomy is a major operation and, at present, almost always an emergency procedure with concomitant high risk of massive blood loss and significant maternal morbidity. Indeed, most maternal mortality occurs during or after hysterectomy.

Overall, the management of patients with severe obstetrical haemorrhage unresponsive to conventional therapy remains unsatisfactory in terms of controlling the bleeding, morbidity, and prevention of mortality. Thus, the introduction of novel haemostatic drugs that are safe and capable of controlling the coagulopathic component and thereby reducing the need for transfusion of blood products and decreasing the consequences of bleeding, including the rate of hysterectomy, may reduce mortality and morbidity in SPPH.

The inventors of the present patent application have found that Factor VII polypeptide having increased activity compared to wild-type Factor VIIa, such as V158D/E296V/M298Q-FVIIa might have an evolving role in the management of severe PPH refractory to conventional treatment due to its enhanced pro-coagulant potential and formation of a stable clot. That Factor VII polypeptide having increased activity is more potent than traditional wild-type human Factor VIIa (NovoSeven) may not only help to decrease exposure to blood products, but also to avert PPH-associated hysterectomies if given as an early treatment.

Some Factor VII polypeptide having increased activity compared to wild-type Factor VIIa, such as V158D/E296V/M298Q-FVIIa retains the same binding to tissue factor (TF) as wild-type human Factor VIIa (or endogenous FVIIa). Also some of these Factor VII polypeptides when bound to TF, have the same activity as wild-type human Factor VIIa. However in the absence of TF on the surface of activated platelets these particular Factor VII polypeptides show increased enzymatic activity as compared to wild-type human Factor VIIa. This increased enzymatic activity results in an increased thrombin generation rate, faster clotting and formation of a more stable hemostatic plug. The rate and peak-levels of thrombin activity seem to be important in determining whether hemostasis in a clinical situation will be effective rather than the total amount of thrombin generated. The more rapid and larger thrombin burst obtained with these particular FVIIa polypeptides described above as compared to rFVIIa (NovoSeven®) makes them particularly suitable for treatment of acute critical severe bleeds, like severe PPH.

The main potential safety concerns associated with Factor VII polypeptide having increased activity compared to wild-type Factor VIIa, such as V158D/E296V/M298Q-FVIIa for treatment of PPH include the potential thromboembolic side effects in patients at risk. However, the theoretical thrombogenicity concern should be limited by the specific characteristics (localized thrombin generation, increased activity on activated platelets, increased reactivity with inhibitors (ATIII and TFPI), fast clearance) indicating a localized hemostasis (thrombin generation) at the site of injury and thus minimized risk of systemic effects.

In conclusion the complex coagulopathy and high complication rate seen in patients with intractable PPH, together with the understanding of the rapid onset of effect, the localized mechanism of action and the low risk of thromboembolic complications following administration of Factor VII polypeptide having increased activity compared to wild-type Factor VIIa and in particular V158D/E296V/M298Q-FVIIa in animal models and of rFVIIa in clinical use, suggest that these FVII polypeptides may possibly be a promising adjunctive therapy for control of severe PPH.

In one particular embodiment Factor VII polypeptides having increased activity compared to wild-type Factor VIIa and in particular V158D/E296V/M298Q-FVIIa could be indicated for the treatment of severe postpartum haemorrhage in patients unresponsive to standard obstetrical management, oxytocic drugs and standard blood component therapy prior to major invasive therapy.

Administration of Factor VII polypeptide having increased activity compared to wild-type Factor VIIa and in particular V158D/E296V/M298Q-FVIIa may be beneficial before hysterectomy in a young patient with severe bleeding, or after internal iliac arteries ligation, if bleeding continues. Factor VII polypeptide having increased activity compared to wild-type Factor VIIa and in particular V158D/E296V/M298Q-FVIIa given either or both during and after hysterectomy might have the potential to conserve blood loss, and might reduce maternal morbidity or even maternal mortality. In some embodiments it may be necessary to replace blood components such as platelets and coagulation factors such as FX, prothrombin and fibrinogen prior to the administration of a Factor VII polypeptide having increased activity compared to wild-type Factor VIIa, such as V158D/E296V/M298Q-FVIIa in severely-depleted coagulopathic women. Earlier treatment with these FVII polypeptides, before deterioration in the condition and coagulation system of the patient seems to be rational, and may reduce bleeding-related mortality and morbidity, as well as complications associated with massive transfusions.

In cases of diffuse bleeding refractory to transfusion therapy and uterotonic agents, use of Factor VII polypeptide having increased activity compared to wild-type Factor VIIa, such as V158D/E296V/M298Q-FVIIa would seem appropriate. Given that many obstetric hospitals do not have on-site interventional radiology, the use of Factor VII polypeptide having increased activity compared to wild-type Factor VIIa, such as V158D/E296V/M298Q-FVIIa may allow additional time to transfer the patient to a hospital where such facilities exist.

When Factor VII polypeptide having increased activity compared to wild-type Factor VIIa, such as V158D/E296V/M298Q-FVIIa has a more rapid onset of effect as well as an improved safety profile then there would be a potential advantage in the use of these FVII polypeptides in the hyper-acute setting of severe PPH.

In a different set of embodiments, the bleeding is due to mild to moderate hemophilia A, where the patient has an endogenous plasma level of FVIII. In some embodiments the bleeding is due to mild hemophilia A. In some embodiments the bleeding is due to moderate hemophilia A.

As used herein moderate hemophilia A is characterized by factor VIII blood plasma levels 0.01-0.05 IU mL⁻¹. Mild hemophilia A is characterized by factor VIII blood plasma levels >0.05<0.40 IU mL⁻¹.

### Factor VII Polypeptide Having Increased Activity Compared To Wild-Type Factor VIIa

In practicing the present invention, any Factor VII polypeptide having increased activity compared to wild-type Factor VIIa may be used that is effective in treating a bleeding episode in a subject with severe bleedings.

The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa.

As used herein, “wild type human FVIIa” is a polypeptide having the amino acid sequence disclosed in U.S. Pat. No. 4,784,950.

Factor VII polypeptide having increased activity compared to wild-type Factor VIIa may include, without limitation, Factor VII polypeptides that have either been chemically modified relative to human Factor VIIa and/or contain one or more amino acid sequence alterations relative to human Factor VIIa. Such Factor VII polypeptides may also apart from activity exhibit other different properties relative to human Factor VIIa, including stability, phospholipid binding, and the like. This includes FVII variants, Factor VII-related polypeptides, Factor VII derivatives and Factor VII conjugates exhibiting increased activity relative to wild-type human Factor VIIa.

The term “Factor VII derivative” as used herein, is intended to designate a FVII polypeptides exhibiting increased activity relative to wild-type Factor VII, in which one or more of the amino acids of the parent peptide have been genetically and/or chemically and/or enzymatically modified, e.g. by alkylation, glycosylation, PEGylation, acylation, ester formation or amide formation or the like. This includes but is not limited to PEGylated human Factor VIIa, cysteine-PEGylated human Factor VIIa and variants thereof.

The term “increased activity” refers to FVII polypeptides with i) increased proteolytic activity compared to recombinant wild type human Factor VIIa or ii) to FVII polypeptides with increased TF binding activity compared to recombinant wild type human Factor VIIa or iii) to FVII polypeptides with increased half life in blood plasma compared to recombinant wild type human Factor VIIa. The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

For purposes of the invention, Factor VIIa proteolytic activity may be quantified by measuring the ability of a preparation to promote blood clotting using Factor VII-deficient plasma and thromboplastin, as described, e.g., in U.S. Pat. No. 5,997,864. In this assay, proteolytic activity is expressed as the reduction in clotting time relative to a control sample and is converted to “Factor VII units” by comparison with a pooled human serum standard containing 1 unit/ml Factor VII activity. Alternatively, Factor VIIa proteolytic activity may be quantified by (i) measuring the ability of Factor VII polypeptide having increased activity compared to wild-type Factor VIIa to produce of Factor Xa in a system comprising TF embedded in a lipid membrane and Factor X. (Persson et al., J. Biol. Chem. 272:19919-19924, 1997); (ii) measuring Factor X hydrolysis in an aqueous system (see “In Vitro Proteolysis Assay”, Example 3 below); (iii) measuring the physical binding of Factor VII polypeptide having increased activity compared to wild-type Factor VIIa to TF using an instrument based on surface plasmon resonance (Persson, FEBS Letts. 413:359-363, 1997) and (iv) measuring hydrolysis of a synthetic substrate by Factor VII polypeptide having increased activity compared to wild-type Factor VIIa (see “In Vitro Hydrolysis Assay”, Example 2 below).

In a further embodiment of the invention, the factor VII polypeptide is a polypeptide, wherein the ratio between the activity of the Factor VII polypeptide and the activity of the wild-type Factor VIIa polypeptide shown in SEQ ID NO:1 is at least about 1.25. In one embodiment the ratio between the activity of the Factor VII polypeptide and the activity of the wild-type Factor VIIa polypeptide shown in SEQ ID NO:1 is at least about 2.0. In a further embodiment the ratio between the activity of the Factor VII polypeptide and the activity of the wild-type Factor VIIa polypeptide shown in SEQ ID NO:1 is at least about 4.0.

In a further embodiment of the invention, the factor VII polypeptide is a polypeptide, wherein the ratio between the activity of the Factor VII polypeptide and the activity of the wild-type Factor VIIa polypeptide shown in SEQ ID NO:1 is at least about 1.25 when tested in a Factor VIIa activity assay. In one embodiment the ratio between the activity of the Factor VII polypeptide and the activity of the wild-type Factor VIIa polypeptide shown in SEQ ID NO:1 is at least about 2.0 when tested in a Factor VIIa activity assay. In a further embodiment the ratio between the activity of the Factor VII polypeptide and the activity of the wild-type Factor VIIa polypeptide shown in SEQ ID NO:1 is at least about 4.0 when tested in a Factor VIIa activity assay. The Factor VIIa activity may be measured by the assays described in examples 2 or 3.

In a further embodiment of the invention, the factor VII polypeptide is a polypeptide, wherein the ratio between the activity of the Factor VII polypeptide and the activity of the wild-type Factor VIIa polypeptide shown in SEQ ID NO:1 is at least about 1.25 when tested in the “In Vitro Hydrolysis Assay”. In one embodiment the ratio between the activity of the Factor VII polypeptide and the activity of the wild-type Factor VIIa polypeptide shown in SEQ ID NO:1 is at least about 2.0 when tested in the “In Vitro Hydrolysis Assay”. In a further embodiment the ratio between the activity of the Factor VII polypeptide and the activity of the wild-type Factor VIIa polypeptide shown in SEQ ID NO:1 is at least about 4.0 when tested in the “In Vitro Hydrolysis Assay”.

In a further embodiment of the invention, the factor VII polypeptide is a polypeptide, wherein the ratio between the activity of the Factor VII polypeptide and the activity of the wild-type Factor VIIa polypeptide shown in SEQ ID NO:1 is at least about 1.25 when tested in the “In Vitro Proteolysis Assay”. In one embodiment the ratio between the activity of the Factor VII polypeptide and the activity of the wild-type Factor VIIa polypeptide shown in SEQ ID NO:1 is at least about 2.0 when tested in the “In Vitro Proteolysis Assay”. In a further embodiment the ratio between the activity of the Factor VII polypeptide and the activity of the wild-type Factor VIIa polypeptide shown in SEQ ID NO:1 is at least about 4.0 when tested in the “In Vitro Proteolysis Assay”. In a further embodiment the ratio between the activity of the Factor VII polypeptide and the activity of the wild-type Factor VIIa polypeptide shown in SEQ ID NO:1 is at least about 8.0 when tested in the “In Vitro Proteolysis Assay”.

Examples of Factor VII polypeptides having increased activity compared to wild-type Factor VIIa, without limitation, wild-type human Factor VIIa, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V1 58D/M298Q/K337A/S314E-FVII, F374Y/L305V/E296V/M 298Q/K337A/V1 58T/S314E-FVII, F374Y/L305V/V158D/E296V/M298Q/K337A/S314E-FVII, S52A-Factor VII, S60A-Factor VII; R152E-Factor VII, S344A-Factor VII, Factor VIIa lacking the Gla domain; and P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys.

Other examples of factor VII equivalents include, without limitation Factor VII equivalents having substantially the same biological activity as wild-type Factor VII including S52A-FVIIa, S60A-FVIIa (Lino et al., Arch. Biochem. Biophys. 352: 182-192, 1998); FVIIa equivalents exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII equivalents as disclosed in WO 02/29025; and FVII equivalents exhibiting increased proteolytic stability as disclosed in WO 02/38162 (Scripps Research Institute); FVII equivalents having a modified Gla-domain and exhibiting an enhanced membrane binding as disclosed in WO 99/20767, WO 00/66753, WO 02/02764, and US patent application 20030211094 (University of Minnesota); and FVII equivalents as disclosed in WO 01/04287, WO 01/58935, WO 03/93465, and US patent application 20030165996 (Maxygen ApS).

Other examples of factor VII equivalents include GlycoPegylated FVII derivatives as disclosed in WO 03/31464 and US Patent applications US 20040043446, US 20040063911, US 20040142856, US 20040137557, and US 20040132640 (Neose Technologies, Inc.).

Non-limiting examples of FVII variants having increased biological activity compared to wild-type FVIIa include FVII variants as disclosed in WO 01/83725, WO 02/22776, WO 02/077218, WO 03/027147, WO 04/029090, WO 05/075635, European patent application with application number 05108713.8 (Novo Nordisk A/S), WO 02/38162 (Scripps Research Institute); and FVIIa variants with enhanced activity as disclosed in JP 2001061479 (Chemo-Sero-Therapeutic Res Inst.).

In one embodiment of the invention, the factor VII polypeptide is K337A-FVII.

In a further embodiment of the invention, the factor VII polypeptide is V158D-FVII.

In a further embodiment of the invention, the factor VII polypeptide is E296V-FVII.

In a further embodiment of the invention, the factor VII polypeptide is M298Q-FVII.

In a further embodiment of the invention, the factor VII polypeptide is V158T-FVII.

In a further embodiment of the invention, the factor VII polypeptide is S314E-FVII.

In a further embodiment of the invention, the factor VII polypeptide is L305V-FVII.

In a further embodiment of the invention, the factor VII polypeptide is L305V/K337A-FVII.

In a further embodiment of the invention, the factor VII polypeptide is L305V/V158D-FVII.

In a further embodiment of the invention, the factor VII polypeptide is L305V/E296V-FVII.

In a further embodiment of the invention, the factor VII polypeptide is L305V/M298Q-FVII.

In a further embodiment of the invention, the factor VII polypeptide is L305V/V158T-FVII.

In a further embodiment of the invention, the factor VII polypeptide is L305V/S314E-FVII.

In a further embodiment of the invention, the factor VII polypeptide is K337A/S314E-FVII.

In a further embodiment of the invention, the factor VII polypeptide is K337A/V158T-FVII.

In a further embodiment of the invention, the factor VII polypeptide is K337A/M 298Q-FVII.

In a further embodiment of the invention, the factor VII polypeptide is K337A/E296V-FVII.

In a further embodiment of the invention, the factor VII polypeptide is K337A/V158D-FVII.

In a further embodiment of the invention, the factor VII polypeptide is V158D/S314E-FVII.

In a further embodiment of the invention, the factor VII polypeptide is V158D/M298Q-FVII.

In a further embodiment of the invention, the factor VII polypeptide is V158D/E296V-FVII.

In a further embodiment of the invention, the factor VII polypeptide is V158T/S314E-FVII.

In a further embodiment of the invention, the factor VII polypeptide is V158T/M298Q-FVII.

In a further embodiment of the invention, the factor VII polypeptide is V158T/E296V-FVII.

In a further embodiment of the invention, the factor VII polypeptide is E296V/S314E-FVII.

In a further embodiment of the invention, the factor VII polypeptide is S314E/M298Q-FVII.

In a further embodiment of the invention, the factor VII polypeptide is E296V/M298Q-FVII.

In a further embodiment of the invention, the factor VII polypeptide is L305V/K337A/V158D-FVII.

In a further embodiment of the invention, the factor VII polypeptide is L305V/K337A/E296V-FVII.

In a further embodiment of the invention, the factor VII polypeptide is L305V/K337A/M298Q-FVII.

In a further embodiment of the invention, the factor VII polypeptide is L305V/K337A/V158T-FVII.

In a further embodiment of the invention, the factor VII polypeptide is L305V/K337A/S314E-FVII.

In a further embodiment of the invention, the factor VII polypeptide is L305V/V158D/E296V-FVII.

In a further embodiment of the invention, the factor VII polypeptide is L305V/V158D/M298Q-FVII.

In a further embodiment of the invention, the factor VII polypeptide is L305V/V158D/S314E-FVII.

In a further embodiment of the invention, the factor VII polypeptide is L305V/E296V/M298Q-FVII.

In a further embodiment of the invention, the factor VII polypeptide is L305V/E296V/V158T-FVII.

In a further embodiment of the invention, the factor VII polypeptide is L305V/E296V/S314E-FVII.

In a further embodiment of the invention, the factor VII polypeptide is L305V/M298Q/V158T-FVII.

In a further embodiment of the invention, the factor VII polypeptide is L305V/M298Q/S314E-FVII.

In a further embodiment of the invention, the factor VII polypeptide is L305V/V158T/S314E-FVII.

In a further embodiment of the invention, the factor VII polypeptide is K337A/S314E/V158T-FVII.

In a further embodiment of the invention, the factor VII polypeptide is K337A/S314E/M298Q-FVII.

In a further embodiment of the invention, the factor VII polypeptide is K337A/S314E/E296V-FVII.

In a further embodiment of the invention, the factor VII polypeptide is K337A/S314E/V158D-FVII.

In a further embodiment of the invention, the factor VII polypeptide is K337A/V158T/M298Q-FVII.

In a further embodiment of the invention, the factor VII polypeptide is K337A/V158T/E296V-FVII.

In a further embodiment of the invention, the factor VII polypeptide is K337A/M298Q/E296V-FVII.

In a further embodiment of the invention, the factor VII polypeptide is K337A/M298Q/V158D-FVII.

In a further embodiment of the invention, the factor VII polypeptide is K337A/E296V/V158D-FVII.

In a further embodiment of the invention, the factor VII polypeptide is V158D/S314E/M298Q-FVII.

In a further embodiment of the invention, the factor VII polypeptide is V158D/S314E/E296V-FVII.

In a further embodiment of the invention, the factor VII polypeptide is V158D/M298Q/E296V-FVII.

In a further embodiment of the invention, the factor VII polypeptide is V158T/S314E/E296V-FVII.

In a further embodiment of the invention, the factor VII polypeptide is V158T/S314E/M298Q-FVII.

In a further embodiment of the invention, the factor VII polypeptide is V158T/M298Q/E296V-FVII.

In a further embodiment of the invention, the factor VII polypeptide is E296V/S314E/M298Q-FVII.

In a further embodiment of the invention, the factor VII polypeptide is L305V/M298Q/K337A/S314E-FVII.

In a further embodiment of the invention, the factor VII polypeptide is L305V/E296V/K337A/S314E-FVII.

In a further embodiment of the invention, the factor VII polypeptide is E296V/M298Q/K337A/S314E-FVII.

In a further embodiment of the invention, the factor VII polypeptide is L305V/E296V/M298Q/K337A-FVII.

In a further embodiment of the invention, the factor VII polypeptide is L305V/E296V/M298Q/S314E-FVII.

In a further embodiment of the invention, the factor VII polypeptide is V158D/E296V/M298Q/K337A-FVII.

In a further embodiment of the invention, the factor VII polypeptide is V158D/E296V/M298Q/S314E-FVII.

In a further embodiment of the invention, the factor VII polypeptide is L305V/V158D/K337A/S314E-FVII.

In a further embodiment of the invention, the factor VII polypeptide is V158D/M298Q/K337A/S314E-FVII.

In a further embodiment of the invention, the factor VII polypeptide is V158D/E296V/K337A/S314E-FVII.

In a further embodiment of the invention, the factor VII polypeptide is L305V/V158D/E296V/M298Q-FVII.

In a further embodiment of the invention, the factor VII polypeptide is L305V/V158D/M298Q/K337A-FVII.

In a further embodiment of the invention, the factor VII polypeptide is L305V/V158D/E296V/K337A-FVII.

In a further embodiment of the invention, the factor VII polypeptide is L305V/V158D/M298Q/S314E-FVII.

In a further embodiment of the invention, the factor VII polypeptide is L305V/V158D/E296V/S314E-FVII.

In a further embodiment of the invention, the factor VII polypeptide is V158T/E296V/M298Q/K337A-FVII.

In a further embodiment of the invention, the factor VII polypeptide is V158T/E296V/M298Q/S314E-FVII.

In a further embodiment of the invention, the factor VII polypeptide is L305V/V158T/K337A/S314E-FVII.

In a further embodiment of the invention, the factor VII polypeptide is V158T/M298Q/K337A/S314E-FVII.

In a further embodiment of the invention, the factor VII polypeptide is V158T/E296V/K337A/S314E-FVII.

In a further embodiment of the invention, the factor VII polypeptide is L305V/V158T/E296V/M298Q-FVII.

In a further embodiment of the invention, the factor VII polypeptide is L305V/V158T/M298Q/K337A-FVII.

In a further embodiment of the invention, the factor VII polypeptide is L305V/V158T/E296V/K337A-FVII.

In a further embodiment of the invention, the factor VII polypeptide is L305V/V158T/M298Q/S314E-FVII.

In a further embodiment of the invention, the factor VII polypeptide is L305V/V158T/E296V/S314E-FVII.

In a further embodiment of the invention, the factor VII polypeptide is E296V/M298Q/K337A/V158T/S314E-FVII.

In a further embodiment of the invention, the factor VII polypeptide is V158D/E296V/M298Q/K337A/S314E-FVII.

In a further embodiment of the invention, the factor VII polypeptide is L305V/V158D/E296V/M298Q/S314E-FVII.

In a further embodiment of the invention, the factor VII polypeptide is L305V/E296V/M298Q/V158T/S314E-FVII.

In a further embodiment of the invention, the factor VII polypeptide is L305V/E296V/M298Q/K337A/V158T-FVII.

In a further embodiment of the invention, the factor VII polypeptide is L305V/E296V/K337A/V158T/S314E-FVII.

In a further embodiment of the invention, the factor VII polypeptide is L305V/M298Q/K337A/V158T/S314E-FVII.

In a further embodiment of the invention, the factor VII polypeptide is L305V/V158D/E296V/M298Q/K337A-FVII.

In a further embodiment of the invention, the factor VII polypeptide is L305V/V158D/E296V/K337A/S314E-FVII.

In a further embodiment of the invention, the factor VII polypeptide is L305V/V158D/M298Q/K337A/S314E-FVII.

In a further embodiment of the invention, the factor VII polypeptide is L305V/E296V/M298Q/K337A/V158T/5314E-FVII

In a further embodiment of the invention, the factor VII polypeptide is L305V/V158D/E296V/M298Q/K337A/S314E-FVII

### Preparations And Formulations

The present invention encompasses therapeutic administration of Factor VII polypeptide having increased activity compared to wild-type Factor VIIas, which is achieved using formulations that comprise Factor VIIa preparations. As used herein, a “Factor VII preparation” refers to a plurality of Factor VIIa polypeptides, including variants and chemically modified forms, that have been separated from the cell in which they were synthesized, whether a cell of origin or a recombinant cell that has been programmed to synthesize Factor VII polypeptide having increased activity compared to wild-type Factor VIIa.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J.-C. Janson and Lars Ryden, editors, VCH Publishers, New York, 1989. Following purification, the preparation preferably contains less than about 10% by weight, more preferably less than about 5% and most preferably less than about 1%, of non-Factor VII proteins derived from the host cell.

Factor VII and Factor VII-related polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII may be activated by passing it through an ion-exchange chromatography column, such as Mono Q® (Pharmacia) or the like. The resulting activated Factor VII may then be formulated and administered as described below.

Pharmaceutical compositions or formulations for use in the present invention comprise a Factor VIIa preparation in combination with, preferably dissolved in, a pharmaceutically acceptable carrier, preferably an aqueous carrier or diluent. A variety of aqueous carriers may be used, such as water, buffered water, 0.4% saline, 0.3% glycine and the like. The preparations of the invention can also be formulated into liposome preparations for delivery or targeting to the sites of injury. Liposome preparations are generally described in, e.g., U.S. Pat. Nos. 4,837,028, 4,501,728, and 4,975,282. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

### Treatment Regimen

In practicing the present invention, Factor VII polypeptide having increased activity compared to wild-type Factor VIIa may be administered to a subject as a single dose comprising a single-dose-effective amount for treating the bleeding episodes in a subject with severe bleedings, or in a staged series of doses which together comprise an effective amount for treating the bleeding episodes in a subject with severe bleedings. An effective amount of Factor VII polypeptide having increased activity compared to wild-type Factor VIIa (see below) refers to the amount of Factor VIIa polypeptide which, when administered in a single dose or in the aggregate of multiple doses, or as part of any other type of defined treatment regimen, produces a measurable improvement in at least one clinical parameter associated with severe bleedings. When Factor VIIa polypeptides with different activity are administered, an effective amount may be determined by comparing the coagulant activity of the new Factor VIIa polypeptides with that of known Factor VIIa polypeptides and adjusting the amount to be administered proportionately to the predetermined effective dose for the known Factor VIIa polypeptides.

Administration of Factor VII polypeptide having increased activity compared to wild-type Factor VIIa according to the present invention is usually initiated within about 6 hours after occurrence of the bleeding episode in a subject with severe bleeding, such as, e.g., within about 4 hours, within about 2 hours, or within about 1 hour. Alternatively, administration may be initiated at any time before start of surgery in subjects in need of such surgery, such as within about 6 hours before surgery, such as, e.g., within about 4 hours, within about 2 hours, or within about 1 hour before surgery e.g., immediately before surgery.

Administration of a single dose refers to administration of an entire dose of Factor VII polypeptide having increased activity compared to wild-type Factor VIIa as a bolus over a period of less than about 5 minutes. In some embodiments, the administration occurs over a period of less than about 2.5 minutes, and, in some, over less than about 1 min.

Typically, a single-dose effective amount comprises at least about 1 μg/kg of a Factor VII polypeptide, such as at least about 10 μg/kg, such as at least about 20 μg/kg, such as at least about 40 μg/kg human Factor VII polypeptide having increased activity compared to wild-type Factor VIIa, such as, at least about 50 μg/kg, 75 μg/kg, or 90 μg/kg, or at least 150 μg/kg Factor VIIa.

In other embodiments, a single-dose effective amount comprises not more than about 1 μg/kg of a Factor VII polypeptide, not more than about 5 μg/kg, such as not more than about 10 μg/kg, such as not more than about 20 μg/kg human Factor VII polypeptide having increased activity compared to wild-type Factor VII, such as, not more than 30 μg/kg, 50 μg/kg, or 75 μg/kg, or not more than 100 μg/kg Factor VIIa.

In some embodiments, following administration of a single dose of Factor VII polypeptide having increased activity compared to wild-type Factor VIIa according to the invention, the subject receives no further Factor VII polypeptide having increased activity compared to wild-type Factor VIIa for an interval of at least about 30 minutes. In some embodiments the post-administration interval is at least about 45 minutes, such as at least about 1 hour, at least about 1.5 hours, or at least about 2 hours.

In other embodiments, the subject receives Factor VII polypeptide having increased activity compared to wild-type Factor VIIa according to the following regimen: (i) The subject receives a first amount of Factor VII polypeptide having increased activity compared to wild-type Factor VIIa comprising at least about 1 μg/kg of a Factor VII polypeptide, such as at least about 10 μg/kg, such as at least about 20 μg/kg, such as at least about 40 μg/kg; (ii) after a period of at least about 30 minutes, a second amount of Factor VII polypeptide having increased activity compared to wild-type Factor VIIa is administered, the amount comprising at least about 1 μg/kg of a Factor VII polypeptide, such as at least about 10 μg/kg, such as at least about 20 μg/kg, such as at least about 40 μg/kg; and (iii) after a period of at least about 30 minutes from administration of the second dose, a third amount of Factor VII polypeptide having increased activity compared to wild-type Factor VIIa is administered, the amount comprising at least about 1 μg/kg of a Factor VII polypeptide, such as at least about 10 μg/kg, such as at least about 20 μg/kg, such as at least about 40 μg/kg. After a period of at least about 30 minutes following the administration of the third amount, the subject may then receive a further (fourth) amount of Factor VII polypeptide having increased activity compared to wild-type Factor VIIa comprising at least about 1 μg/kg of a Factor VII polypeptide, such as at least about 10 μg/kg, such as at least about 20 μg/kg, such as at least about 40 μg/kg.

In other embodiments, the first amount of Factor VII polypeptide having increased activity compared to wild-type Factor VIIa comprises at least about 1 μg/kg of a Factor VII polypeptide, such as at least about 10 μg/kg, such as at least about 20 μg/kg, such as at least about 40 μg/kg, such as at least about 80 μg/kg, such as at least about 100 μg/kg or at least about 150 μg/kg; in other embodiments, the second amount of Factor VII polypeptide having increased activity compared to wild-type Factor VIIa comprises at least about 75 μg/kg, such as at least about 90 μg/kg; in other embodiments, the third (and optionally fourth) amount of a Factor VII polypeptide having increased activity compared to wild-type Factor VIIa comprises at least about 75 μg/kg, such as at least about 90 μg/kg.

In other embodiments, the subject receives Factor VII polypeptide having increased activity compared to wild-type Factor VIIa according to the following regimen: (i) The subject receives a first amount of Factor VII polypeptide having increased activity compared to wild-type Factor VIIa comprising not more than about 1 μg/kg of a Factor VII polypeptide, such as not more than about 10 μg/kg, such as not more than about 20 μg/kg, such as not more than about 40 μg/kg; (ii) after a period of at least about 30 minutes, a second amount of Factor VII polypeptide having increased activity compared to wild-type Factor VIIa is administered, the amount comprising not more than about 1 μg/kg of a Factor VII polypeptide, such as not more than about 10 μg/kg, such as not more than about 20 μg/kg, such as not more than about 40 μg/kg; and (iii) after a period of at least about 30 minutes from administration of the second dose, a third amount of Factor VII polypeptide having increased activity compared to wild-type Factor VIIa is administered, the amount comprising not more than about 1 μg/kg of a Factor VII polypeptide, such as not more than about 10 μg/kg, such as not more than about 20 μg/kg, such as not more than about 40 μg/kg. After a period of at least about 30 minutes following the administration of the third amount, the subject may then receive a further (fourth) amount of Factor VII polypeptide having increased activity compared to wild-type Factor VIIa comprising not more than about 1 μg/kg of a Factor VII polypeptide, such as not more than about 10 μg/kg, such as not more than about 20 μg/kg, such as not more than about 40 μg/kg.

In other embodiments, the first amount of Factor VII polypeptide having increased activity compared to wild-type Factor VIIa comprises not more than about 1 μg/kg of a Factor VII polypeptide, such as not more than about 10 μg/kg, such as not more than about 20 μg/kg, such as not more than about 40 μg/kg, such as not more than about 80 μg/kg, such as not more than about 100 μg/kg or not more than about 150 μg/kg; in other embodiments, the second amount of Factor VII polypeptide having increased activity compared to wild-type Factor VIIa comprises not more than about 75 μg/kg, such as not more than about 90 μg/kg; in other embodiments, the third (and optionally fourth) amount of a Factor VII polypeptide having increased activity compared to wild-type Factor VIIa comprises not more than about 75 μg/kg, such as not more than about 90 μg/kg.

In one embodiment, the first dose comprises about 200 μg/kg, the second dose about 100 μg/kg, and the third (and optionally fourth) dose about 100 μg/kg.

In other embodiments, the subject receives the second amount of a Factor VII polypeptide having increased activity compared to wild-type Factor VIIa after a period of at least about 45 minutes from the first administration, such as at least about 1 hour, at least about 1.5 hours, at least about 2 hours, at least about 2.5 hours, or at least about 3 hours.

In other embodiments, the subject receives the third (and optionally fourth) amount of Factor VII polypeptide having increased activity compared to wild-type Factor VIIa after a period of at least about 45 minutes from the previous administration, such as at least about 1 hour, at least about 1.5 hours, at least about 2 hours, at least about 2.5 hours, or at least about 3 hours.

In one embodiment, the subject receives a first dose comprising about 200 μg/kg; after a period of about 1 hour, the subject receives a second dose comprising about 100 μg/kg, and after a period of about 3 hours from the first dose, the subject receives a third dose comprising about 100 μg/kg.

The following table illustrates different non-limiting embodiments of the invention:

TABLE 1

| Time post- | | | | | | | | | | | |
|---|---|---|---|---|---|---|---|---|---|---|---|
| injury or | | | | | | | | | | | |
| post | | | Time to | | 2^{nd} dose | | Time to | | 3^{rd} Dose | | Additional |
| excision | 1^{st} Dose | | 2^{nd} dose | | (optional) | | 3^{rd} dose | | (optional) | | Doses |
| 0-≦1 h | >1 | mcg/kg | 0-1 | h | >1 | mcg/kg | 0-1 | h | >1 | mcg/kg | As needed |
| | | | 1-2 | h | | | 1-2 | h | | | |
| | | | ≧3 | h | | | ≧3 | h | | | |
| | >40 | mcg/kg | 0-1 | h | >40 | mcg/kg | 0-1 | h | >40 | mcg/kg | |
| | | | 1-2 | h | | | 1-2 | h | | | |
| | | | ≧3 | h | | | ≧3 | h | | | |
| | 100 | mcg/kg | 0-1 | h | 50-100 | mcg/kg | 0-1 | h | 50-100 | mcg/kg | |
| | | | 1-2 | h | | | 1-2 | h | | | |
| | | | ≧3 | h | | | ≧3 | h | | | |
| | ≧150 | mcg/kg | 0-1 | h | 50-100 | mcg/kg | 0-1 | h | 50-100 | mcg/kg | |
| | | | 1-2 | h | | | 1-2 | h | | | |
| | | | ≧3 | h | | | ≧3 | h | | | |
| >1-≦3 h | >1 | mcg/kg | 0-1 | h | >1 | mcg/kg | 0-1 | h | >1 | mcg/kg | As needed |
| | | | 1-2 | h | | | 1-2 | h | | | |
| | | | ≧3 | h | | | ≧3 | h | | | |
| | >40 | mcg/kg | 0-1 | h | >40 | mcg/kg | 0-1 | h | >40 | mcg/kg | |
| | | | 1-2 | h | | | 1-2 | h | | | |
| | | | ≧3 | h | | | ≧3 | h | | | |
| | 100 | mcg/kg | 0-1 | h | 50-100 | mcg/kg | 0-1 | h | 50-100 | mcg/kg | |
| | | | 1-2 | h | | | 1-2 | h | | | |
| | | | ≧3 | h | | | ≧3 | h | | | |
| | ≧150 | mcg/kg | 0-1 | h | 50-100 | mcg/kg | 0-1 | h | 50-100 | mcg/kg | |
| | | | 1-2 | h | | | 1-2 | h | | | |
| | | | ≧3 | h | | | ≧3 | h | | | |
| >3-<6 h | >1 | mcg/kg | 0-1 | h | >1 | mcg/kg | 0-1 | h | >1 | mcg/kg | As needed |
| | | | 1-2 | h | | | 1-2 | h | | | |
| | | | ≧3 | h | | | ≧3 | h | | | |
| | >50 | mcg/kg | 0-1 | h | >50 | mcg/kg | 0-1 | h | >50 | mcg/kg | |
| | | | 1-2 | h | | | 1-2 | h | | | |
| | | | ≧3 | h | | | ≧3 | h | | | |
| | 100 | mcg/kg | 0-1 | h | 50-100 | mcg/kg | 0-1 | h | 50-100 | mcg/kg | |
| | | | 1-2 | h | | | 1-2 | h | | | |
| | | | ≧3 | h | | | ≧3 | h | | | |
| | ≧150 | mcg/kg | 0-1 | h | 50-100 | mcg/kg | 0-1 | h | 50-100 | mcg/kg | |
| | | | 1-2 | h | | | 1-2 | h | | | |
| | | | ≧3 | h | | | ≧3 | h | | | |
| >6-<12 h | >1 | mcg/kg | 0-1 | h | >1 | mcg/kg | 0-1 | h | >1 | mcg/kg | As needed |
| | | | 1-2 | h | | | 1-2 | h | | | |
| | | | ≧3 | h | | | ≧3 | h | | | |
| | >50 | mcg/kg | 0-1 | h | >50 | mcg/kg | 0-1 | h | >50 | mcg/kg | |
| | | | 1-2 | h | | | 1-2 | h | | | |
| | | | ≧3 | h | | | ≧3 | h | | | |
| | 100 | mcg/kg | 0-1 | h | 50-100 | mcg/kg | 0-1 | h | 50-100 | mcg/kg | |
| | | | 1-2 | h | | | 1-2 | h | | | |
| | | | ≧3 | h | | | ≧3 | h | | | |
| | ≧150 | mcg/kg | 0-1 | h | 50-100 | mcg/kg | 0-1 | h | 50-100 | mcg/kg | |
| | | | 1-2 | h | | | 1-2 | h | | | |
| | | | ≧3 | h | | | ≧3 | h | | | |

It will be understood that the effective amount of Factor VII polypeptide having increased activity compared to wild-type Factor VIIa, as well as the overall dosage regimen, may vary according to the subject's haemostatic status, which, in turn, may be reflected in one or more clinical parameters, including, e.g., relative levels of circulating coagulation factors; amount of blood lost; rate of bleeding; hematocrit, and the like. It will be further understood that the effective amount may be determined by those of ordinary skill in the art by routine experimentation, by constructing a matrix of values and testing different points in the matrix.

For example, in one series of embodiments, the invention encompasses (i) administering a first dose of a Factor VII polypeptide having increased activity compared to wild-type Factor VIIa; (ii) assessing the subject's coagulation status after a predetermined time; and (iii) based on the assessment, administering a further dose of Factor VII polypeptide having increased activity compared to wild-type Factor VIIa if necessary. Steps (ii) and (iii) may be repeated until satisfactory hemostasis is achieved.

According to the invention, a Factor VII polypeptide having increased activity compared to wild-type Factor VIIa may be administered by any effective route, including, without limitation, intravenous, intramuscular, subcutaneous, mucosal, and pulmonary routes of administration. In one embodiment administration is by an intravenous route. In one embodiment administration is by subcutaneous route.

### Combination Treatments

The present invention encompasses combined administration of an additional agent in concert with a Factor VII polypeptide having increased activity compared to wild-type Factor VIIa. In some embodiments, the additional agent comprises a coagulant, including, without limitation, a coagulation factor such as, e.g. Factor V (see, e.g., PCT/DK02/00736), Factor VIII, Factor IX (see, e.g., WO 02/062376), Factor X, Factor XI, Factor XIII (see, e.g., WO 01/85198), Fibrinogen, thrombin, TAFI (see, e.g., PCT/DK02/00734), Antifibrinolytics such as, e.g., PAI-1, aprotinin, epsilon-aminocaproic acid or tranexamic acid (see, e.g., PCT/DK02/00735; PCT/DK02/00742; PCT/DK02/00751; PCT/DK02/00752);, various anti-thrombotic treatments, as well as transfusions with platelet, RBC, FFP, oxygen carriers, the various bypassing agents and fluid therapies (colloids/crystalloids) or any combination thereof; inhibitors of tissue factor pathway inhibitor (TFPI inhibitors) (see, e.g., WO 01/85199); protein C inhibitors (see, e.g., PCT/DK02/00737); thrombomodulin (see, e.g., PCT/DK02/00738); protein S inhibitors (see, e.g., PCT/DK02/00739); tissue plasminogen activator inhibitors (see, e.g., PCT/DK02/00740); α2-antiplasmin (see, e.g., PCT/DK02/00741);

It will be understood that, in embodiments comprising administration of combinations of Factor VIIa with other agents, the dosage of Factor VII polypeptide having increased activity compared to wild-type Factor VIIa may on its own comprise an effective amount and additional agent(s) may further augment the therapeutic benefit to the subject. Alternatively, the combination of Factor VIIa or equivalent and the second agent may together comprise an effective amount for treating the bleeding episodes in a subject with severe bleedings. It will also be understood that effective amounts may be defined in the context of particular treatment regimens, including, e.g., timing and number of administrations, modes of administrations, formulations, etc.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

### BRIEF DESCRIPTION OF THE DRAWINGS

FIG. 1 shows the full amino acid sequence of native (wild type) human coagulation Factor VII (SEQ ID NO:1).

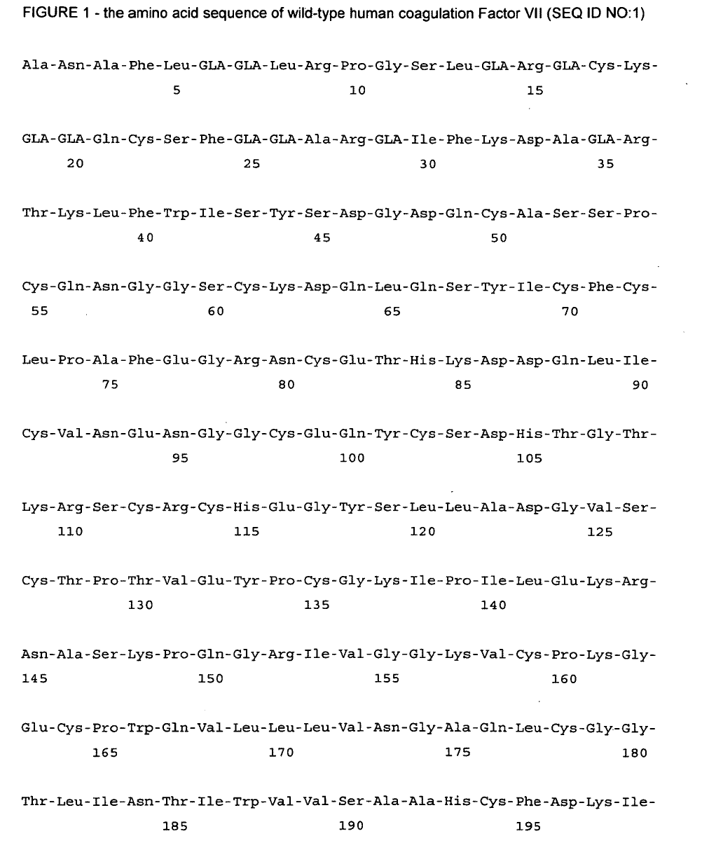

FIG. 2 shows the effect of rFVIIa and V158D/E296V/M298Q-FVIIa in blood mimicking an anticoagulant condition where whole blood was spiked with physiological concentrations of Innohep; A represents Innohep+25 nM V158D/E296V/M298Q-FVIIa; B represents HWB+buffer; C represents Innohep+buffer, D represents 2.5 nM V158D/E296V/M298Q-FVIIa, and E represents 25 nM rFVIIa.

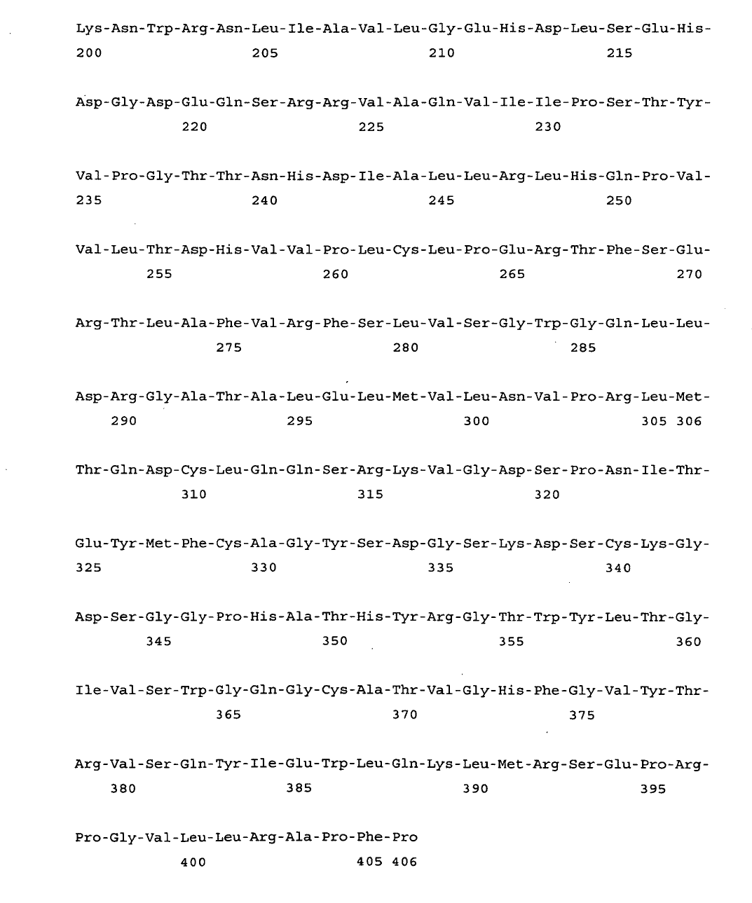

FIG. 3 shows effect of wild-type human FVIIa and V158D/E296V/M298Q-FVIIa in a Mild to Moderate hemophilia A model. V158D/E296V/M298Q-FVIIa is represented in the Fig as “analog factor VIIa” or “analog”, wild-type human FVIIa as “VIIa”.

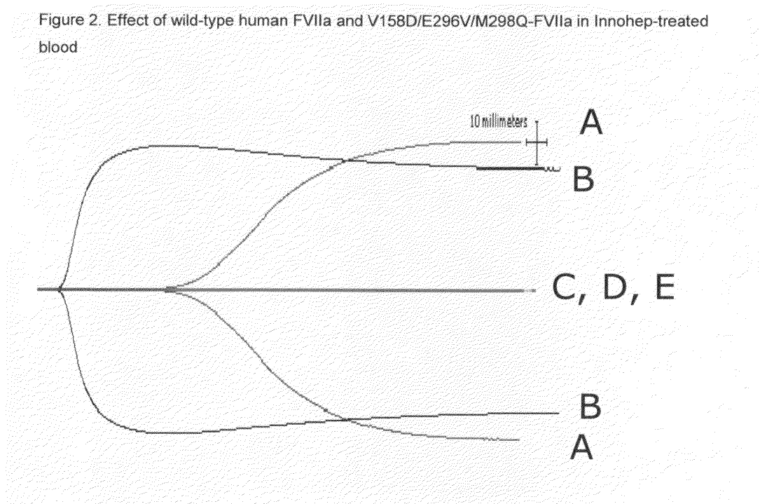

### EXAMPLES

### Example 1

The terminology for amino acid substitutions used the following examples are as follows. The first letter represent the amino acid naturally present at a position of SEQ ID NO:1. The following number represent the position in SEQ ID NO:1. The second letter represent the different amino acid substituting for (replacing) the natural amino acid. An example is M298Q, where an methionine at position 298 of SEQ ID NO:1 is replaced by a glutamine. In another example, V158T/M298Q, the valine in position 158 of SEQ ID NO:1 is replaced by a threonine and the methionine in position 298 of SEQ ID NO:1 is replaced by a Glutamine in the same Factor VII polypeptide.

FVIIa polypeptides having increased activity compared to wild-type Factor VIIa to be used according to the invention may be prepared according to published international patent applications, e.g. WO 01/83725, WO 02/22776, WO 02/077218, WO 03/027147, WO 04/029090, WO 05/075635, European patent application with application number 05108713.8 (Novo Nordisk A/S), WO 02/38162 and JP 2001061479.

### Example 2

### In Vitro Hydrolysis Assay

Native (wild-type) Factor VIIa and Factor VIIa variant (both hereafter referred to as “Factor VIIa”) are assayed in parallel to directly compare their specific activities. The assay is carried out in a microtiter plate (MaxiSorp, Nunc, Denmark). The chromogenic substrate D-Ile-Pro-Arg-p-nitroanilide (S-2288, Chromogenix, Sweden), final concentration 1 mM, is added to Factor VIIa (final concentration 100 nM) in 50 mM Hepes, pH 7.4, containing 0.1 M NaCl, 5 mM CaCl2 and 1 mg/ml bovine serum albumin. The absorbance at 405 nm is meas-ured continuously in a SpectraMax™ 340 plate reader (Molecular Devices, USA). The absorbance developed during a 20-minute incubation, after subtraction of the absorbance in a blank well containing no enzyme, is used to calculate the ratio between the activities of vari-ant and wild-type Factor VIIa:

Ratio=(A405 nm Factor VIIa variant)/(A405 nm Factor VIIa wild-type).

### Example 3

### In Vitro Proteolysis Assay

Native (wild-type) Factor VIIa and Factor VIIa variant (both hereafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 microM) in 100 microL 50 mM Hepes, pH 7.4, containing 0.1 M NaCl, 5 mM CaCl2 and 1 mg/ml bovine serum albumin, are incubated for 15 min. Factor X cleavage is then stopped by the addition of 50 microL 50 mM Hepes, pH 7.4, containing 0.1 M NaCl, 20 mM EDTA and 1 mg/ml bovine serum albumin. The amount of Factor Xa generated is measured by addition of the chromogenic substrate Z-D-Arg-Gly-Arg-p-nitroanilide (S-2765, Chromogenix, Swe-den), final concentration 0.5 mM. The absorbance at 405 nm is measured continuously in a SpectraMax™ 340 plate reader (Molecular Devices, USA). The absorbance developed dur-ing 10 minutes, after subtraction of the absorbance in a blank well containing no FVIIa, is used to calculate the ratio between the proteolytic activities of variant and wild-type Factor VIIa:

Ratio=(A405 nm Factor VIIa variant)/(A405 nm Factor VIIa wild-type).

### Example 4

The present example demonstrates the impact of low pH and temperature as well as crystalloid and colloid hemodilution on the effect of wild type recombinant FVIIa (rwtFVIIa) (25 nM≈90 ug/kg) and V158D/E296V/M298Q-FVIIA (25 nM≈90 ug/kg), upon clot formation as measured by TEG using a low amount of tissue factor (Innovin, final dilution 1:42500) and citrate stabilized whole blood (WB) from healthy volunteers (normal values n=12, hemodiluted values n=6).

The efficacy of V158D/E296V/M298Q-FVIIA has also been tested in animal models of trauma (rabbits and pig models).

Experimental hemodilution, acidosis and hypothermia were created by 40% dilution with either a crystalloid (Ringers lactate) or a colloid solution as 5% albumin, HES130/0.4 (Voluven®), HES 200/0.5 (Haes-Steril®), 6% Hetastarch or Hextend. In addition, hemodilution was combined with a pH at 7.0 (adjusted with a 1M HEPES buffer) and a temperature at 32° C. (extreme conditions).

Under certain in vitro conditions wild type recombinant FVIIa (rwtFVIIa) showed no significant effect when blood was hemodiluted combined with pH 7 and low temperature (32 C) mimicking trauma conditions whereas V158D/E296V/M298Q-FVIIa demonstrated significant effect

TABLE 2

| | CFR (Angle) | | |
|---|---|---|---|
| | | | V158D/E296V/ |
| | No | rwtFVIIa, | M298Q- |
| | addition | 25 nM | FVIIa, 25 nM |
| HWB (n = 12) | 51.6 | 59.6 | 67.8 |
| RL(40%) | 64.6 | 65.4 | 70.1 |
| RL(40%) pH 7 32° C. | 56.8 | 64.6 | 67.7 |
| HSA 5%(40%) | 53.2 | 59.8 | 64.3 |
| HSA 5%(40%) pH 7 32° C. | 45.0 | 54.2 | 61.2 |
| HES 200(40%) | 50.9 | 48.0 | 58.4 |
| HES 200(40%) pH 7 32° C. | 45.3 | 48.0 | 56.7 |
| HES 130(40%) | 48.5 | 49.2 | 55.7 |
| HES 130(40%) pH 7 32° C. | 43.0 | 44.3 | 53.3 |
| Hetastarch(40%) | 42.5 | 41.1 | 49.9 |
| Hetastarch(40%) pH 7 32° C. | 42.7 | 36.5 | 47.5 |
| Hextend(40%) | 43.3 | 44.5 | 50.9 |
| Hextend(40%) pH 7 32° C. | 41.3 | 41.8 | 49.9 |

Dilution with and without low pH and low temperature induce a hypocoagulable state (however, angle was only significantly impaired under extreme hemodilution conditions).

rwtFVIIa was not able to improve the angle (velocity of clot formation) in conditions where the angle was significant reduced, whereas V158D/E296V/M298Q-FVIIa significantly improved the angle.

Thus, V158D/E296V/M298Q-FVIIA demonstrated significant and superior effect compared to the effect obtained by rwtFVIIa mimicking in vitro trauma conditions. V158D/E296V/M298Q-FVIIa is superior to rwtFVIIa in inducing hemostasis under in vitro conditions mimicking trauma.

### Example 5

We have investigated the in vitro effect of rFVIIa (25 nM) and V158D/E296V/M298Q-FVIIa (2.5 nM and 25 nM) in blood mimicking an anticoagulant condition where whole blood was spiked with physiological concentrations of Melagatran, Arixtra, Abciximab (Reopro), Integrillin, Heparin or Innohep.
For all thromboelastography (TEG 5000 analyser, Haemoscope corporation) experiments citrate stabilized whole blood (WB) was used and the coagulation was initiated with tis-sue factor (Innovin, final dilution 1:42500). Initial different whole blood concentrations for the anti coagulants were investigated using TEG ranging from 0.01-0.25 ug/ml Mela-gatran, 0.05-1.5 ug/ml Arixtra, 2.5-50 ug/mI Abciximab (Reopro), 2.5-50 ug/ml In-tegrillin, 0.05-0.5 ie/ml Heparin or 0.05-0.5 ie/ml Innohep. Following the titration studies, experiments were performed using whole blood from 8 healthy donors with ex vivo addi-tion of two different concentrations of V158D/E296V/M298Q-FVIIa and one concentration of rFVIIa combined with ex vivo spiking with one physiology dose of Melagatran (0.2 ug/ml), Arixtra (0.5 ug/ml), Abciximab (Reopro) (10 ug/mI), Integrillin (10 ug/ml), Heparin (0.5 ie/ml) or Innohep (0.5 ie/ml). The statistical analysis was performed by a two-way ANOVA model. P<0.05 was considered statistically significant. Statistical data was not included for Heparin and Innohep since only TEG traces were observed by ex vivo adding 25 nM V158D/E296V/M298Q-FVIIa.
The effect of rFVIIa and V158D/E296V/M298Q-FVIIa on human whole blood showed a significantly reduced R-time, increased MA and MTG, however rFVIIa did not significantly increased the angle (clot formation time) as V158D/E296V/M298Q-FVIIA did. Dose response of V158D/E296V/M298Q-FVIIa was observed for all anti coagulants
Treatment of human whole blood with Heparin or Innohep 0.5 ie/ml did not formed a clot and treatment with 25 nM rFVIIa or 2.5 nM V158D/E296V/M298Q-FVIIa had no effect on clot formation whereas 25 nM V158D/E296V/M298Q-FVIIa was able to generate a clot. V158D/E296V/M298Q-FVIIA demonstrated significant and superior effect compared to the effect obtained by rFVIIa in whole blood mimicking an anticoagulant treatment such as heparin.

TABLE 3
Effect of rFVIIa and V158D/E296V/M298Q-FVIIa in Innohep-treated blood

| | R-Time | α- angle | MTG (mm/sec × | MA |
|---|---|---|---|---|
| | (Sec) | (deg) | 100) | (mm) |
| HWB + buffer | 486 ± 64 | 55.5 ± 3.9 | 11.8 ± 1.7 | 60.7 ± 4.7 |
| Innohep | 0 | 0 | 0 | 0 |
| Innohep + 2.5 nM | 0 | 0 | 0 | 0 |
| V158D/E296V/M298Q- | | | | |
| FVIIa | | | | |
| Innohep + 25 nM | 1522 ± 622 | 23.2 ± 6.2 | 4.5 ± 1.2 | 58.9 ± 6 |
| V158D/E296V/M298Q- | | | | |
| FVIIa | | | | |
| Innohep + 25 nM | 0 | 0 | 0 | 0 |
| rFVIIa | | | | |

### Example 6

### Effect of Wild-Type Human FVIIa And V158D/E296V/M298Q-FVIIa In A Mild To Moderate Hemophilia A Model, FVIII Activation By rFVIIa

Thrombin generation experiment: Thrombinoscope BV system were used for TG assay using plasma. According to the commercial PPP reagent, the sensitivity of the assay is poor. Therefore, we ellagic acid (fc 2 uM) were added. Synthetic phospholipid, PC/PS/PE (fc 16.5 uM), TF(fc 6.7 pM) is used. According to this modified TG assay, dose-dependent pattern can be observed with very low level of FVIII (0.1-1.0%of FVIII activity in FVIII deficient plasma).
Results of the Thrombin generation assay:

| | | HA plasma + Wild-type | | | | | HA plasma + | | | | |
|---|---|---|---|---|---|---|---|---|---|---|---|
| | NP | human FVIIa | | | | | V158D/E296V/M298Q-FVIIa | | | | |
| uM | | 0 | 0.05 | 0.1 | 0.2 | 0.4 uM | 0 | 0.05 | 0.1 | 0.2 | 0.4 uM |
| Lagtime | 17 | 0 | 12.2 | 11.5 | 9 | 8.83 | 0 | 5.33 | 3.83 | 2.67 | 2 |
| Peak | 389 | 0 | 45.2 | 45.9 | 47.2 | 96.2 | 0 | 175 | 217 | 241 | 230 |
| ttPeak | 20 | 0 | 48.5 | 37.3 | 27 | 27.7 | 0 | 14.3 | 9.83 | 7 | 5 |

Measurement of FVIII activity: Factor VIII(a) activity was measured in a one-stage clotting assay using factor VIII-deficient plasma (FIG. 4). All reactions were performed at 37° C. All factor VIII products were incubated in buffer (20 mM HEPES, pH 7.2, 0.1 M NaCl, 5 mM CaCl2, 100 mg/ml BSA, and 0.01% Tween 20) plus the indicated concentrations of PL and TF. Samples were removed from the mixtures at the indicated times and were immediately diluted by several hundred-fold at 4° C. The presence of wild-type human factor VIIa or V158D/E296V/M298Q-FVIIa diluted sample was shown not to affect in the coagulation assay.

### Example 7

### Effect of Wild-Type Human FVIIa And V158D/E296V/M298Q-FVIIa On Clopidogrel-Induced Bleeding In Rats

Clopidogrel (Plavix; Sanofi Aventis) is an irreversible ADP receptor antagonist, which effectively inhibits platelet aggregation. Clopidogrel is extensively used for prevention of thromboembolic events e.g. myocardial infarctions. One of the potential adverse events following clopidogrel treatment is uncontrolled bleeding, e.g. if acute surgical intervention is needed. Currently, no effective antidote for clopidogrel exists.

The hypothesis of the present study was that wild-type human FVIIa, which currently are registered for use against bleeding in inhibitor-complicated haemophilia, may be used to treat bleeding caused by clopidogrel. Thus, the effect of wild-type human FVIIa and, V158D/E296V/M298Q-FVIIa, a new potent rFVIIa-analogue, was tested in a tail-bleeding model in rats pretreated with clopidogrel.

Rats were dosed orally with 10 mg/kg clopidogrel. After 4 hours, tail-transsection was performed. Five minutes after, the initiation of the bleeding, the rats were treated with wild-type human FVIIa (5, 10, 20 mg/kg), V158D/E296V/M298Q-FVIIa (2, 5, 10 mg/kg) or vehicle, where after the blood loss was determined in the following 30 minutes.

TABLE 4
Blood loss (nmol hemglobin; n = 10)

| | Group | wild-type human FVIIa | V158D/E296V/M298Q-FVIIa |
|---|---|---|---|
| A | Normal rats | 510 ± 465 | |
| B | Clopidogrel + vehicle | 13933 ± 2434 *** vs. A | |
| C | Clopidogrel + 2 mg/kg wild-type | nd | 6703 ± 2097 |
| | human FVIIa/V158D/E296V/M298Q- | | |
| | FVIIa | | |
| D | Clopidogrel + 5 mg/kg wild-type | 8573 ± 2199^{£} | 3031 ± 1164** vs. B |
| | human FVIIa/V158D/E296V/M298Q- | | |
| | FVIIa | | |
| E | Clopidogrel + 10 mg/kg wild-type | 3867 ± 1468** vs. B^{$} | 1241 ± 1044*** vs. B^{$} |
| | human FVIIa/V158D/E296V/M298Q- | | |
| | FVIIa | | |
| F | Clopidogrel + 20 mg/kg wild-type | 3112 ± 678** vs. B^{$} | nd |
| | human FVIIa/V158D/E296V/M298Q- | | |
| | FVIIa | | |

Data are mean ± SEM. Data are analyzed after square root transformation using Student's t-test (A-B) or one-way ANOVA with Bonferroni's post-test (B-F).
Asterisks indicate statistical significance at:
**p < 0.01 and
***p < 0.001.
nd: not determined.
^{£}n = 8;
^{$}n = 9

Clopidogrel significantly increased the blood loss compared to normal rats (p<0.001). This increase in blood loss was significantly and dose-dependently reduced by both wild-type human FVIIa and V158D/E296V/M298Q-FVIIa. The dose-response curves were fitted to a sigmoidal curve, showing a higher potency of V158D/E296V/M298Q-FVIIa compared to wild-type human FVIIa. An observed higher maximum effect of V158D/E296V/M298Q-FVIIa compared to wild-type human FVIIa, may indicate a higher efficacy of V158D/E296V/M298Q-FVIIa.

### Example 8

### Effect of Wild-Type Human FVIIa And V158D/E296V/M298Q-FVIIa On Tail-Bleeding In Thrombocytopenic Rats

Thrombocytopenia may be caused by a number of underlying diseases, and may be the cause of uncontrolled bleeding. Currently, the standard therapy against thrombocytopenic bleeding is platelet transfusion. The aim of the present study was to examine the effect of wild-type human FVIIa and V158D/E296V/M298Q-FVIIa, an rFVIIa-analogue with increased potency, in rats with antibody-induced thrombocytopenia.

Rats received a subcutaneous injection of polyclonal rabbit anti-rat thrombocyte-antibody.

(Accurate Chemicals, Westbury, N.Y.). After 24 hours, when the tail transsection was performed, the platelet number was reduced with more than 90% of the initial value 589±185 to 46±21×10⁹/l; n=58; mean±SD). After 5 minutes of bleeding, rats were treated with wild-type human FVIIa (5 or 10 mg/kg), V158D/E296V/M298Q-FVIIa (10 mg/kg) or vehicle, where after the bleeding was observed for 1800 seconds.

TABLE 5
Total bleeding time and blood loss

| | Group | Total bleeding time (s) | Blood loss (nmol hemglobin/ml) |
|---|---|---|---|
| A | Normal rats (n = 10) | 91 ± 44 | 4.0 ± 4.0 |
| B | Thrombocytopenia + vehicle (n = 12) | 1436 ± 161 ***vs. A | 78.7 ± 22.7 ***vs. A |
| C | Thrombocytop. + 5 mg/kg wild-type | 896 ± 200 | 46.8 ± 24.6 |
| | human FVIIa (n = 12) | | |
| D | Thrombocytop. + 10 mg/kg wild-type | 310 ± 67 **vs. B | 15.2 ± 9.7 |
| | human FVIIa (n = 11) | | |
| E | Thrombocytop. + 10 mg/kg | 550 ± 207 **vs. B | 0 ± 0 ^{#} **vs. B |
| | V158D/E296V/M298Q-FVIIa (n = 11) | | |

Data are mean ± SEM. Bleeding time data are analyzed using Mann-Whitneys U-test (A-B) or Kruskall-Wallis test with Dunn's post-test (B-E). Blood loss data are analyzed after log(x + 1) transformation using Student's t-test (A-B) or one-way ANOVA with Bonferroni's post-test (B-E).
Asterisks indicate statistical significance at:
** p < 0.01 and
*** p < 0.001.
^{#} below detection limit.

wild-type human FVIIa (10 mg/kg) caused a significant reduction in bleeding time, whereas a numerical reduction in blood loss did not reach statistical significance (table 5). V158D/E296V/M298Q-FVIIa reduced both bleeding time and blood loss significantly. In fact, no blood loss, detected as haemoglobin concentration, was detected in V158D/E296V/M298Q-FVIIa treated animals, indicating that the observed bleeding time, was due to oozing of plasma components rather than erythrocytes.

In conclusion, both wild-type human FVIIa and V158D/E296V/M298Q-FVIIa had an effect on tail-bleeding in thrombocytopenic rats with less than 10% of the normal platelet counts. This study indicates that V158D/E296V/M298Q-FVIIa may have a beneficial effect in the treatment of bleeding episodes caused by a severe reduction in the level thrombocytes.

### Example 9

### Effect of Wild-Type Human FVIIa And V158D/E296V/M298Q-FVIIa On Low Molecular Weight Heparin-Induced Bleeding In Rats

The aim of the present experiment was to compare the effect of wild-type human FVIIa and V158D/E296V/M298Q-FVIIa, an rFVIIa-analogue with increased potency, in rats anticoagulated with two different doses of tinzaparin. Thus, rats received 500 or 1800 IU/kg of tinzaparin intravenously. After 10 minutes tail cut was performed, and after another 5 minutes rats were treated with 20 mg/kg wild-type human FVIIa, 10 mg/kg V158D/E296V/M298Q-FVIIa or vehicle, where after the bleeding was observed for 1800 seconds.

TABLE 6
Total bleeding time (s)

| | Group | Tinzaparin (500 IU/kg; n = 9) | Tinzaparin (1800 IU/kg; n = 10) |
|---|---|---|---|
| A | No tinzaparin | 542 ± 180 (n = 9) | |
| B | Tinzaparin + Vehicle | 1800 ± 0 ***vs. A | 1800 ± 0 |
| C | Tinzaparin + 20 mg/kg wild-type | 1038 ± 206 *vs. B | 1357 ± 123 NS vs. B |
| | human FVIIa | | |
| D | Tinzaparin + 10 mg/kg | 404 ± 143 ***vs. B | 463 ± 92 ***vs. B; *vs. C |
| | V158D/E296V/M298Q-FVIIa | | |

Data are mean ± SEM. Data are analyzed using Mann-Whitneys U-test (A-B) or Kruskall-Wallis test with Dunn's post-test (B-D).
Asterisks indicate statistical significance at:
* p < 0.05 and
*** p < 0.001.
^{#} observation period 1800 s.

TABLE 7
Blood loss (nmol hemoglobin)

| | Group | Tinzaparin (500 IU/kg; n = 9) | Tinzaparin (1800 IU/kg; n = 10) |
|---|---|---|---|
| A | No tinzaparin | 316 ± 316 (n = 9) | |
| B | Tinzaparin + Vehicle | 9836 ± 2415 ***vs. A | 13179 ± 3048 |
| C | Tinzaparin + 20 mg/kg wild-type | 1470 ± 614 *vs. B | 4383 ± 1050 NS vs. B |
| | human FVIIa | | |
| D | Tinzaparin + 10 mg/kg | 0 ± 0 ^{#} ***vs. B | 351 ± 149 ***vs. B & C |
| | V158D/E296V/M298Q-FVIIa | | |

Data are mean ± SEM. Data are analyzed after log(x + 1) transformation using Student's t-test (A-B) or one-way ANOVA with Bonferroni's post-test (B-E).
Asterisks indicate statistical significance at:
* p < 0.05 and
*** p < 0.001.
^{#} below detection limit
NS: non-significant

wild-type human FVIIa (20 mg/kg) caused a significant reduction in bleeding time and blood loss during a bleeding induced by 500 IU/kg tinzaparin (table 6 and 7, left column), while, V158D/E296V/M298Q-FVIIa was capable to normalize both bleeding time and blood loss.

After severe anticoagulation with 1800 IU/kg tinzaparin, a highly significant effect of 10 mg/kg V158D/E296V/M298Q-FVIIa (10 mg/kg) on bleeding time and blood loss compared to the vehicle control group was retained (table 6 and 7, right column). In contrast, a two-fold higher dose of wild-type human FVIIa (20 mg/kg) did not affect bleeding time or blood loss significantly, although a numerical reduction was observed in both variables. This difference in effect between V158D/E296V/M298Q-FVIIa and wild-type human FVIIa reached statistical significance for bleeding time as well as blood loss.

In conclusion, both wild-type human FVIIa and V158D/E296V/M298Q-FVIIa had an effect on tinzaparin-induced bleeding in rats, but under severely anticoagulated animals V158D/E296V/M298Q-FVIIa seemed more efficacious than wild-type human FVIIa.

### Embodiments of the Invention

1. Use of a Factor VII polypeptide having increased activity compared to wild-type Factor VIIa for the manufacture of a medicament for the treatment of bleeding episodes in a subject with severe bleedings.
2. Use according to any of embodiment 1, wherein the ratio between the activity of said Factor VII polypeptide and the activity of the wild-type Factor VIIa polypeptide shown in SEQ ID NO:1 is at least about 1.25.
3. The Factor VII polypeptide according to embodiment 2, wherein said ratio is at least about 2.0, such as at least about 4.0, such as at least about 6, such as at least about 10.
4. Use according to any one of embodiments 1 to 3, wherein the medicament comprises at least about 1 μg/kg of a Factor VII polypeptide, such as at least about 10 μg/kg, such as at least about 20 μg/kg, such as at least about 40 μg/kg, such as at least about 80 μg/kg, such as at least about 100 μg/kg of a Factor VII polypeptide having increased activity compared to wild-type Factor VIIa.
5. Use according to any one of embodiments 1 to 4, wherein the medicament is for administration in a first dose containing at least about 1 μg/kg of a Factor VII polypeptide, such as at least about 10 μg/kg, such as at least about 20 μg/kg, such as at least about 40 μg/kg, such as at least about 80 μg/kg of a Factor VII polypeptide having increased activity compared to wild-type Factor VIIa, followed by a second dose containing at least about 1 μg/kg of a Factor VII polypeptide, such as at least about 10 μg/kg, such as at least about 20 μg/kg, such as at least about 40 μg/kg, such as at least about 80 μg/kg of a Factor VII polypeptide having increased activity compared to wild-type Factor VIIa administered one to 24 hours after the start of treatment.
6. Use according to embodiment 5, wherein a further, third dose containing at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa is administered at least about one hour after the start of the second treatment.
7. Use according to any one of embodiments 1 to 6, wherein the subject with severe bleeding experience acidosis with a pH lower than 7.45, such as lower than 7.35, such as lower than 7.25, such as lower than 7.15, such as lower than 7.05, such as lower than 6.95, such as lower than 6.85.
8. Use according to any one of embodiments 1 to 7, wherein the subject with severe bleeding experience hypothermia with a temperature below 37 degree Celsius, such as below 36 degree celsius, such as below 35 degree celsius, such as below 34 degree celsius, such as below 33 degree celsius, such as below 32 degree celsius, such as below 31 degree celsius, such as below 30 degree celsius.
9. Use according to any one of embodiments 1 to 8, wherein the subject with severe bleeding experience pre-treatment haemoglobin level 4 g/dl lower than the normal level for said subject, such as 5 g/dl lower than the normal level for said subject, such as 6 g/dl lower than the normal level for said subject, such as 7 g/dl lower than the normal level for said subject, such as 8 g/dl lower than the normal level for said subject, such as 9 g/dl lower than the normal level for said subject, such as 10 g/dl lower than the normal level for said subject, such as 11 g/dl lower than the normal level for said subject, such as 12 g/dl lower than the normal level for said subject, such as 13 g/dl lower than the normal level for said subject.
10. Use according to any one of embodiments 1 to 9, wherein the subject with severe bleeding experience pre-treatment haemoglobin level lower than 14 g/dl, such as a pre-treatment haemoglobin level lower than 12 g/dl, such as a pre-treatment haemoglobin level lower than 10 g/dl, such as a pre-treatment haemoglobin level lower than 8 g/dl, such as a pre-treatment haemoglobin level lower than 6 g/dl, such as a pre-treatment haemoglobin level lower than 4 g/dl, such as a pre-treatment haemoglobin level lower than 3 g/dl, such as a pre-treatment haemoglobin level lower than 2 g/dl.
11. Use according to any one of embodiments 1 to 10, wherein the subject with severe bleeding requires fluid replacement therapy of 1000 ml crystalloids or colloids, such as a fluid replacement therapy of 1500 ml crystalloids or colloids, such as a fluid replacement therapy of 2000 ml crystalloids or colloids, such as a fluid replacement therapy of 2500 ml crystalloids or colloids, such as a fluid replacement therapy of 3000 ml crystalloids or colloids, such as a fluid replacement therapy of 3500 ml crystalloids or colloids, such as a fluid replacement therapy of 4000 ml crystalloids or colloids, such as a fluid replacement therapy of 4500 ml crystalloids or colloids, such as a fluid replacement therapy of 5000 ml crystalloids or colloids, such as a fluid replacement therapy of 6000 ml crystalloids or colloids.
12. Use according to any one of embodiments 1 to 11, wherein the medicament further comprises a second coagulation agent in an amount that augments said preventing or attenuating by said Factor VII polypeptide having increased activity compared to wild-type Factor VIIa.
13. Use according to embodiment 12, wherein said second coagulation agent is selected from the group consisting of a coagulation factor and an antifibrinolytic agent.
14. Use according to embodiment 13, wherein said coagulation agent is selected from the group consisting of Factor V, Factor VIII, Factor IX, Factor X, Factor XI, Factor XIII, Fibrinogen, thrombin, TAFI, PAI-1, aprotinin, epsilon-aminocaproic acid or tranexamic acid, various antithrombotic treatments, as well as transfusions with platelet, RBC, FFP, oxygen carriers, the various bypassing agents and fluid therapies (colloids/crystalloids).
15. Use according to any one of embodiments 1 to 14, wherein said Factor VII polypeptide having increased activity compared to wild-type Factor VIIa is V158D/E296V/M298Q-FVIIa.
16. Kit of parts for the treatment of bleeding episodes in a subject with severe bleedings, comprising
(i) A medicament comprising a Factor VII polypeptide having increased activity compared to wild-type Factor VIIa; and
(ii) Instructions for Use describing that:
a. A first dose containing at least about 1 μg/kg of a Factor VII polypeptide, such as at least about 10 μg/kg, such as at least about 20 μg/kg, such as at least about 40 μg/kg, such as at least about 80, such as at least about 100 μg/kg Factor VII polypeptide having increased activity compared to wild-type Factor VIIa, should be administered at the start of treatment;
b. Optionally, a second dose containing at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa should be administered one to 24 hours after the start of treatment.
17. Kit according to embodiment 16, wherein the instructions for use further describes that an optional third dose containing at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa may be administered to said subject at least about one hour after the start of the second treatment.
18. A method for the treatment of bleeding episodes in a subject with severe bleedings, the method comprising administering to a subject in need of said treatment an effective amount for said treatment of a Factor VII polypeptide having increased activity compared to wild-type Factor VIIa.
19. A method according to embodiment 18, wherein the subject with severe bleeding experience acidosis with a pH lower than 7.45, such as lower than 7.35, such as lower than 7.25, such as lower than 7.15, such as lower than 7.05, such as lower than 6.95, such as lower than 6.85.
20. A method according to any one of embodiments 18 to 19, wherein the subject with severe bleeding experience hypothermia with a temperature below 37 degree Celsius, such as below 36 degree celsius, such as below 35 degree celsius, such as below 34 degree celsius, such as below 33 degree celsius, such as below 32 degree celsius, such as below 31 degree celsius, such as below 30 degree celsius.
21. A method according to any one of embodiments 18 to 20, wherein the subject with severe bleeding experience pre-treatment haemoglobin level 4 g/dl lower than the normal level for said subject, such as 5 g/dl lower than the normal level for said subject, such as 6 g/dl lower than the normal level for said subject, such as 7 g/dl lower than the normal level for said subject, such as 8 g/dl lower than the normal level for said subject, such as 9 g/dl lower than the normal level for said subject, such as 10 g/dl lower than the normal level for said subject, such as 11 g/dl lower than the normal level for said subject, such as 12 g/dl lower than the normal level for said subject, such as 13 g/dl lower than the normal level for said subject.
22. A method according to any one of embodiments 18 to 21, wherein the subject with severe bleeding experience pre-treatment haemoglobin level lower than 14 g/dl, such as a pre-treatment haemoglobin level lower than 12 g/dl, such as a pre-treatment haemoglobin level lower than 10 g/dl, such as a pre-treatment haemoglobin level lower than 8 g/dl, such as a pre-treatment haemoglobin level lower than 6 g/dl, such as a pre-treatment haemoglobin level lower than 4 g/dl, such as a pre-treatment haemoglobin level lower than 3 g/dl, such as a pre-treatment haemoglobin level lower than 2 g/dl.
23. A method according to any one of embodiments 18 to 22, wherein the subject with severe bleeding requires fluid replacement therapy of 1000 ml crystalloids or colloids, such as a fluid replacement therapy of 1500 ml crystalloids or colloids, such as a fluid replacement therapy of 2000 ml crystalloids or colloids, such as a fluid replacement therapy of 2500 ml crystalloids or colloids, such as a fluid replacement therapy of 3000 ml crystalloids or colloids, such as a fluid replacement therapy of 3500 ml crystalloids or colloids, such as a fluid replacement therapy of 4000 ml crystalloids or colloids, such as a fluid replacement therapy of 4500 ml crystalloids or colloids, such as a fluid replacement therapy of 5000 ml crystalloids or colloids, such as a fluid replacement therapy of 6000 ml crystalloids or colloids.
24. A method according to any one of embodiments 18 to 23, wherein said effective amount comprises at least about 1 μg/kg of a Factor VII polypeptide, such as at least about 10 μg/kg, such as at least about 20 μg/kg, such as at least about 40 μg/kg, such as at least about 80 μg/kg, such as at least about 100 μg/kg of a Factor VII polypeptide having increased activity compared to wild-type Factor VIIa.
25. A method according to any one of embodiments 18 to 24, wherein a first amount of at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa is administered at the start of treatment, and a second amount of at least about 1 μg/kg of a Factor VII polypeptide, such as at least about 10 μg/kg, such as at least about 20 μg/kg, such as at least about 40 μg/kg, such as at least about 80 μg/kg of Factor VII polypeptide having increased activity compared to wild-type Factor VIIa is administered to the subject one to 24 hours after the start of treatment.
26. A method according to embodiment 25, further comprising administering to the subject a third amount of at least about 1 μg/kg of a Factor VII polypeptide, such as at least about 10 μg/kg, such as at least about 20 μg/kg, such as at least about 40 μg/kg, such as at least about 80 μg/kg of Factor VII polypeptide having increased activity compared to wild-type Factor VIIa at least about one hour after the start of the second treatment.
27. A method according to any one of embodiments 18 to 26, further comprising administering to the subject a second coagulation agent in an amount that augments said treating by said Factor VII polypeptide having increased activity compared to wild-type Factor VIIa.
28. A method according to embodiment 27, wherein said second coagulation agent is selected from the group consisting of a coagulation factor and an antifibrinolytic agent.
29. A method according to embodiment 28, wherein said coagulation agent is selected from the group consisting of Factor V, Factor VIII, Factor IX, Factor X, Factor XI, Factor XIII, Fibrinogen, thrombin, TAFI, PAI-1, aprotinin, epsilon-aminocaproic acid or tranexamic acid, various antithrombotic treatments, as well as transfusions with platelet, RBC, FFP, oxygen carriers, the various bypassing agents and fluid therapies (colloids/crystalloids).
30. A method for the treatment of bleeding episodes in a subject with severe bleedings in a majority of subjects with severe bleedings, said method comprising (i) administering to a group of subjects with a severely bleeding an effective amount for said treatment of Factor VII polypeptide having increased activity compared to wild-type Factor VIIa; and (ii) observing an improvement in one or more clinical parameters of said bleeding episode among said group of subjects relative to the level of said clinical parameters that would have been expected in the same group of subjects who had not received said Factor VII polypeptide having increased activity compared to wild-type Factor VIIa.
31. A method according to any one of embodiments 18 to 30, wherein said Factor VII polypeptide having increased activity compared to wild-type Factor VIIa is V158D/E296V/M298Q-FVIIa.

1. A method for the treatment of bleeding episodes in a subject with severe bleedings, the method comprising administering to a subject in need of said treatment an effective amount for said treatment of a Factor VII polypeptide having increased activity compared to wild-type Factor VIIa.

2. A method according to claim 1, wherein the subject with severe bleeding exhibits acidosis with a blood pH lower than 7.45.

3. A method according to claim 1, wherein the subject with severe bleeding exhibits a pre-treatment haemoglobin level at least about 4 g/dl lower than the normal level for said subject.

4. A method according to claim 1, wherein the subject with severe bleeding exhibits hypothermia with a temperature below 37 degree Celsius,

5. A method according to claim 1, wherein the ratio between the activity of said Factor VII polypeptide and the activity of the wild-type Factor VIIa polypeptide shown in SEQ ID NO:1 is at least about 1.25.

6. A method according to claim 5, wherein said Factor VII polypeptide having increased activity compared to wild-type Factor VIIa is V158D/E296V/M298Q-FVIIa.

7. A method according to claim 1, wherein said effective amount comprises not more than about 100 μg/kg of a Factor VII polypeptide.

8. A method according to claim 7, wherein said effective amount comprises not more than about 20 μg/kg of a Factor VII polypeptide.

9. A method according to claim 8, wherein said effective amount comprises not more than about 5 μg/kg of a Factor VII polypeptide.

10. A method according to claim 1, further comprising administering to the subject a second coagulation agent in an amount that augments said treating by said Factor VII polypeptide having increased activity compared to wild-type Factor VIIa.

11. A method according to claim 10, wherein said second coagulation agent is selected from the group consisting of a coagulation factor and an antifibrinolytic agent.

12. A method according to claim 11, wherein said coagulation agent is selected from the group consisting of Factor V, Factor VIII, Factor IX, Factor X, Factor XI, Factor XIII, Fibrinogen, thrombin, TAFI, PAI-, aprotinin, epsilon-aminocaproic acid, tranexamic acid, an antithrombotic treatment, and transfusions with one or more of platelet, RBC, FFP, and oxygen carriers.

13. A method for the treatment of bleeding episodes in a subject with severe bleedings in a majority of subjects with severe bleedings, said method comprising (i) administering to a group of subjects with a severely bleeding an effective amount for said treatment of Factor VII polypeptide having increased activity compared to wild-type Factor VIIa; and (ii) observing an improvement in one or more clinical parameters of said bleeding episode among said group of subjects relative to the level of said clinical parameters that would have been expected in the same group of subjects who had not received said Factor VII polypeptide having increased activity compared to wild-type Factor VIIa.

14. A kit of parts for treatment of bleeding episodes in a subject with sever bleeding, comprising
(i) A medicament comprising a Factor VII polypeptide having increased activity compared to wild-type Factor VIIa; and
(ii) Instructions for Use describing that:
a. A first dose containing no more than about 100 μg/kg of a Factor VII polypeptide having increased activity compared to wild-type Factor VIIa, should be administered at the start of treatment;
b. Optionally, a second dose containing no more than about 100 μg/kg Factor VII polypeptide having increased activity compared to wild-type Factor VIIa should be administered one to 24 hours after the start of treatment.
